(19) United States
(12) Patent Application Publication
(10) Pub. No.: US 2025/0034663 A1
(43) Pub. Date: Jan. 30, 2025

(54) METHOD FOR DETERMINING AAV GENOMES

(71) Applicant: Hoffmann-La Roche Inc., Little Falls, NJ (US)

(72) Inventors: Johannes Auer, Schwaigen (DE); Anna Metzger, Basel (CH); Monika Popp, Polling/Oderding (DE)

(73) Assignee: Hoffmann-La Roche Inc., Little Falls, NJ (US)

(21) Appl. No.: 18/912,379

(22) Filed: Oct. 10, 2024

Related U.S. Application Data
(63) Continuation of application No. PCT/EP2023/059404, filed on Apr. 11, 2023.

(30) Foreign Application Priority Data
Apr. 13, 2022 (EP) 22168080.4

(51) Int. Cl. C12Q 1/70

(52) U.S. Cl. CPC C12Q 1/701; C12Q 2600/156

(57) ABSTRACT

Herein is reported a method for the determination of viral genome DNA copy number in a sample, wherein the method comprises the steps of incubating the sample with proteinase K and determining the viral genome DNA copy number by digital droplet polymerase chain reaction, wherein the sample is free of DNA, which is not encapsidated within a viral particle, wherein the incubation with proteinase K is in the presence of 0.05 (w/v) % to 1.5 (w/v) % sodium dodecyl sulfate.

### CROSS-REFERENCE TO RELATED APPLICATIONS

This application is a continuation of International Application No. PCT/EP2023/059404, filed Apr. 11, 2023, which claims benefit of priority to European Patent Application No. 22168080.4 filed Apr. 13, 2022, each of which is incorporated herein by reference in its entirety.

### FIELD OF INVENTION

The current invention is in the field of gene therapy. In more detail, herein is reported a method for determining viral genome copy numbers in process and purified samples with ddPCR, wherein the sample is incubated prior to the PCR with proteinase K in the presence of a detergent.

### BACKGROUND OF THE INVENTION

With their good safety profile, high therapeutic efficacy and the possibility of target specific engineering, adeno-associated virus (AAV) particles are commonly used as gene transfer vehicles for research and in clinical approaches. For recombinant production, accurate and robust analytical methods for viral vector characterization are required. Commonly, vector genome titration is performed by droplet digital PCR (ddPCR), which is a method for absolute quantification of nucleic acids.

In literature, several pretreatment methods are reported for AAV vector genome titration, for both, real time and droplet digital PCR. Prior to absolute quantification of target sequences within the capsid, free transient plasmids and other unpackaged nucleic acids, like host cell DNA, need to be degraded with nucleases like DNase I (Zolotukhin, S., et al. 1999; Furuta-Hanawa, B., et al. 2019; Fripont, S., et al. 2019; Dobnik, D., et al. 2019; Sanmiguel J., et al. 2019). Furthermore, the capsid needs to be degraded for better vector genome accessibility. Heat inactivation of the AAV particles and the resulting denaturation of its capsid proteins are sufficient for that purpose (Wang, Y., et al. 2019). However, in most publications an additional protein digest with Proteinase K is described (Zolotukhin, S., et al. 1999; Fripont, S., et al. 2019; Dobnik, D., et al. 2019; Sanmiguel J., et al. 2019). Moreover, there are various recommendations for dilution buffers for subsequent dilution series of the treated samples: Besides of nuclease-free water (Zolotukhin, S., et al. 1999), TE buffer (Furuta-Hanawa, B., et al. 2019; Wang, Y., et al. 2019) or PCR buffers (Sanmiguel J., et al. 2019), additives like the anti-surfactant Pluronic F-68 (Furuta-Hanawa, B., et al. 2019; Sanmiguel J., et al. 2019) and sheared salmon sperm (sss) DNA (Lock, M., et al. 2014; Sanmiguel J., et al. 2019) are described in literature.

Suoranta, T., et al. (Hum. Gen. Ther. 32 (2020) 1270-1279) compared extraction of AAV1, AAV2, AAV5, AAV6, AAV8, and AAV9 genomes after iodixanol gradient ultracentrifugation in phosphate buffered saline solution by heat denaturation, proteinase K treatment, and kit extraction using qPCR and ddPCR. Kit extraction, which contained proteinase K treatment in the presence of additional carrier RNA in denaturing buffer before spin-column purification, significantly increased the titers acquired for all the serotypes in both qPCR and ddPCR. Importantly, Suoranta et al. found that no study has presented conclusive data on genome availability in ddPCR.

CN 109957561 discloses a method for extracting nucleic acids from a sample, wherein the method comprises the steps of adding a lysis solution to the sample to be extracted to release nucleic acid molecules; further adding a lauroyl sarcosine sodium salt solution; further adding a mixed solution containing sodium iodide, glycogen and isopropanol, whereby a precipitate containing the nucleic acid is formed which is recovered by centrifuging the sample.

U.S. Pat. No. 8,652,821 discloses a reagent mixture for purifying RNA-free DNA, which comprises a protease, an RNase, and a detergent.

U.S. Pat. No. 11,028,372 discloses a scalable purification method for AAV particles of the serotype rh. 10.

WO 03/104413 discloses a not further specified dot blot analysis of pseudotyped recombinant AAV virions comprising proteinase K incubation followed by phenol extraction and ethanol precipitation.

WO 2007/084773 discloses a not further specified dot blot analysis of infectious parvovirus vectors produced in insect cells.

Binny, C. J. and Nathwani, A. C. disclose an agarose gel analysis method for the determination whether the genome packaged into a scAAV vector is, as intended, a short double-stranded hairpin structure or a short ssDNA or a long, non-folded ssDNA (Meth. Mol. Biol. 891 (2012) 109-131).

US 2021/0284699 discloses a qPCR method for analyzing rAAV particles that have been purified using a method comprising the steps of (a) generating a viral particle extract comprising a plurality of rAAV provided herein, wherein the viral particle extract comprises the supernatant of lysed producer cells, or a derivative thereof; (b) contacting the viral particle extract with an ionic detergent to generate a first mixture; (c) contacting the first mixture with an acid to generate a second mixture; (d) centrifuging the second mixture to generate a supernatant; (e) filtering the supernatant with one or more filters to generate a filtrate; (f) performing one or more cycles of buffer exchange of the filtrate to a final storage buffer.

### SUMMARY OF THE INVENTION

Herein is reported a method for the determination of viral genome DNA copy number in a sample, wherein the method comprises the steps of incubating the sample with proteinase K and determining the viral genome DNA copy number by digital droplet polymerase chain reaction, wherein the sample is free of DNA, which is not encapsidated within a viral particle, wherein the incubation with proteinase K is in the presence of a 0.05 (w/v) % to 1.5 (w/v) % sodium dodecyl sulfate.

A non-limiting number of aspects (independent subject matter) and embodiments (dependent subject matter) is
1. A method for the determination of viral genome DNA copy number in a sample, wherein the method comprises the step of
   determining the viral genome DNA copy number by digital droplet polymerase chain reaction,
wherein the sample is free of DNA, which is not encapsidated within a viral particle,
wherein the sample has not been incubated with a nuclease, and
wherein the sample has not been incubated with a protease.
2. The method according to aspect 1, wherein the nuclease is a restriction enzyme.
3. The method according to any one of aspect 1 or embodiment 2, wherein the nuclease is DNase I.
4. The method according to any one of aspect 1 or embodiments 2 to 3, wherein the protease is proteinase K.
5. A method for the determination of viral genome DNA copy number in a sample, wherein the method comprises the steps of
   incubating the sample with proteinase K,
   determining the viral genome DNA copy number by digital droplet polymerase chain reaction.
6. The method according to aspect 5, wherein the sample is a cell lysate.
7. The method according to any one of aspect 5 or embodiment 6, wherein the sample is a lysed cell sample.
8. The method according to embodiment 7, wherein the lysed cell sample has been obtained by lysing cells producing the virus with a detergent or by chemical means; optionally cell debris has been removed from the lysed cell sample.
9. The method according to any one of aspect 5 or embodiments 6 to 8, wherein the sample comprises viral particles, wherein the viral DNA genome is encapsidated, and free DNA, which is not encapsidated within a viral particle.
10. A method for the determination of viral genome DNA copy number in a sample, wherein the method comprises the steps of
   incubating the sample with proteinase K,
   determining the viral genome DNA copy number by digital droplet polymerase chain reaction, wherein the sample is free of DNA, which is not encapsidated within a viral particle, wherein the incubation with proteinase K is in the presence of a detergent.
11. The method according to aspect 10, wherein the detergent is sodium dodecyl sulfate.
12. The method according to any one of aspect 10 or embodiment 11, wherein the final concentration of the detergent during the incubation with proteinase K is 0.05 (w/v) % to 1.5 (w/v) %
13. The method according to any one of aspect 10 or embodiments 11 to 12, wherein the final concentration of the detergent during the incubation with proteinase K is about 0.1 (w/v) %.
14. The method according to any one of aspect 10 or embodiments 11 to 12, wherein the final concentration of the detergent during the incubation with proteinase K is about 1 (w/v) %.
15. The method according to any one of aspects 5 or aspect 10 or embodiments 6 to 9 or embodiments 11 to 14, wherein the method comprises the following steps
   incubating the sample with a nuclease to obtain a digested sample,
   incubating the digested sample with proteinase K to obtain a proteinase K incubated sample,
   determining the viral genome DNA copy number in the proteinase K incubated sample by digital droplet polymerase chain reaction.
16. The method according to embodiment 15, wherein the digested sample is diluted most 2.5-times for the incubation with proteinase K.
17. The method according to any one of embodiments 15 to 16, wherein the complete digested sample is incubated with proteinase K.
18. The method according to any one of aspects 1 or aspect 5 or aspect 10 or embodiments 2 to 4 or embodiments 6 to 9 or embodiments 11 to 17, wherein the digital droplet polymerase chain reaction comprises the following steps
   a) incubating the sample at about 95° C. for 10 minutes,
   b) performing a thermal cycle of incubating the sample at about 94° C. for 30 seconds followed by incubating the sample at about 60° C. for 1 minute,
   c) repeating step b) for 15 to 60 times,
   d) performing a final elongation step at 98° C. for 10 minutes.
19. The method according to embodiment 18, wherein in steps a) and d) are performed with a temperature ramping of 2° C.
20. The method according to any one of aspects 1 or aspect 5 or aspect 10 or embodiments 2 to 4 or embodiments 6 to 9 or embodiments 11 to 19, wherein the sample is maintained at temperatures of 95° C. or lower.
21. The method according to any one of aspects 1 or aspect 5 or aspect 10 or embodiments 2 to 4 or embodiments 6 to 9 or embodiments 11 to 19, wherein the sample is not exposed to a temperature above 95° C.
22. The method according to any one of aspects 1 or aspect 5 or aspect 10 or embodiments 2 to 4 or embodiments 6 to 9 or embodiments 11 to 19, wherein the method is performed at a temperature of at most 95° C. except for the final elongation step of the digital droplet polymerase chain reaction.
23. The method according to any one of aspect 5 or aspect 10 or embodiments 2 to 4 or embodiments 6 to 9 or embodiments 11 to 22, wherein the total amount of proteinase K employed in the incubation is 1 mU to 50 mU.
24. The method according to any one of aspect 5 or aspect 10 or embodiments 2 to 4 or embodiments 6 to 9 or embodiments 11 to 23, wherein the total amount of proteinase K employed in the incubation is 10 mU to 40 mU.
25. The method according to any one of aspect 5 or aspect 10 or embodiments 2 to 4 or embodiments 6 to 9 or embodiments 11 to 24, wherein the total amount of proteinase K employed in the incubation is 15 mU to 35 mU.
26. The method according to any one of aspect 5 or aspect 10 or embodiments 2 to 4 or embodiments 6 to 9 or embodiments 11 to 25, wherein the total amount of proteinase K employed in the incubation is 20 mU to 32 mU.
27. The method according to any one of aspect 5 or aspect 10 or embodiments 2 to 4 or embodiments 6 to 9 or embodiments 11 to 26, wherein the total amount of proteinase K employed in the incubation is about 20 mU.
28. The method according to any one of aspect 5 or aspect 10 or embodiments 2 to 4 or embodiments 6 to 9 or embodiments 11 to 26, wherein the total amount of proteinase K employed in the incubation is about 32 mU.
29. The method according to any one of aspect 1 or aspect 5 or aspect 10 or embodiments 2 to 4 or embodiments 6 to 9 or embodiments 11 to 28, wherein the determining of the viral genome DNA copy number is a quantifying of the viral genome DNA copy number.
30. The method according to any one of aspect 1 or aspect 5 or aspect 10 or embodiments 2 to 4 or embodiments 6 to 9 or embodiments 11 to 29, wherein the volume of the sample is about 10 μL.
31. The method according to any one of aspect 5 or aspect 10 or embodiments 2 to 4 or embodiments 6 to 9 or embodiments 11 to 30, wherein the incubating with proteinase K is in a total volume of 100 μL.
32. The method according to any one of embodiments 15 to 31, wherein the nuclease is DNase I.
33. The method according to any one of embodiments 15 to 32, wherein the total amount of nuclease employed in the incubation is about 5 U.
34. The method according to any one of embodiments 15 to 33, wherein the incubating with the nuclease is in a total volume of 50 μL.
35. The method according to any one of embodiments 15 to 34, wherein the incubating with the nuclease is at final concentrations of 40 mM Tris*HCl, 10 mM MgSO₄, 1 mM CaCl₂) at a pH value of about 8.
36. The method according to any one of embodiments 15 to 35, wherein the incubating with the nuclease is at 37° C. for 30 minutes.
37. The method according to embodiment 36, wherein the incubating is followed by an inactivating of the nuclease at 95° C. for 15 minutes.
38. The method according to any one of embodiments 15 to 37, wherein the incubating with proteinase K is at final concentrations of 20 mM Tris*HCl, 1 mM EDTA, 100 mM NaCl at a pH value of about 8.
39. The method according to any one of embodiments 15 to 37, wherein the incubating with proteinase K is at final concentrations of 20 mM Tris*HCl, 1 mM EDTA, 100 mM NaCl at a pH value of about 8.
40. The method according to any one of embodiments 15 to 37, wherein the incubating with proteinase K is at final concentrations of 20 mM Tris*HCl, 1 mM EDTA, 100 mM NaCl, 1 (w/v) % sodium dodecyl sulfate at a pH value of about 8.
41. The method according to any one of embodiments 15 to 37, wherein the incubating with proteinase K is at final concentrations of 30 mM Tris*HCl, 5 mM MgSO₄, 0.5 mM CaCl₂), 0.5 mM EDTA, 50 mM NaCl, 0.1 (w/v) % sodium dodecyl sulfate at a pH value of about 8.
42. The method according to any one of embodiments 15 to 41, wherein the incubating with proteinase K is at 50° C. for 60 minutes.
43. The method according to embodiment 42, wherein the incubating is followed by an inactivating of the protease at 95° C. for 15 minutes.
44. The method according to any one of aspect 1 or aspect 5 or aspect 10 or embodiments 2 to 4 or embodiments 7 to 9 or embodiments 11 to 43, wherein the volume of the sample is an affinity chromatography purified cell lysate.
45. The method according to any one of aspect 1 or aspect 5 or aspect 10 or embodiments 2 to 4 or embodiments 6 to 9 or embodiments 11 to 43, wherein the method is performed in the absence of a precipitation step or/and glycogen.

### DETAILED DESCRIPTION OF THE INVENTION

The current invention is based, at least in part, on the finding that for AAV genome copy number determination the sample has to be incubated prior to the PCR with proteinase K in case of crude cell lysate samples comprising AAV particles and additionally comprising not virally encapsidated DNA. The presence of a detergent can further improve the determination.

The current invention is based, at least in part, on the finding that for AAV genome copy number determination the sample has to be incubated prior to the PCR with proteinase K in the presence of a detergent in case of purified samples comprising AAV particles and which are essentially free of not virally encapsidated DNA. Without being bound to this theory, it is assumed that the detergent prevents the formation of aggregates of protein fragments and viral genomic DNA, which prevent the later amplification by polymerase chain reaction, and thereby results in an underestimation of the viral genome copy number in the sample.

The current invention is based, at least in part, on the finding that for AAV genome copy number determination after sequential treatment with a nuclease and a protease the protease treatment has to be carried out in the presence of a detergent. Without being bound to this theory it is assumed that the detergent prevents the formation of aggregates of protein fragments and viral genomic DNA which prevent the later amplification by polymerase chain reaction and thereby underestimation of the viral genome copy number in the sample.

The current invention is further based, at least in part, on the finding that for purified samples comprising AAV particles and which are essentially free of not virally encapsidated DNA the determination of the AAV genome copy number has to be done either without nuclease and proteinase pre-treatment or with proteinase K incubation in the presence of a detergent prior to the polymerase chain reaction. Without being bound by this theory, it is assumed that the incubation of a highly purified sample, i.e. not comprising essential amounts of not virally encapsidated DNA, with a nuclease alone or in combination with a protease in the absence of a detergent results in aggregation and thereby artificially lowered AAV genome copy numbers.

The current invention is further based, at least in part, on the finding that for purified samples comprising AAV particles and which are essentially free of not virally encapsidated DNA the determination of the AAV genome copy number has to be done without a thermal denaturation step at a temperature higher than 95° C. Without being bound by this theory, it is assumed that the thermal treatment of a highly purified sample, i.e. not comprising essential amounts of not virally encapsidated DNA, at temperatures above 95° C. results in aggregation and thereby artificially lowered AAV genome copy numbers.

The invention is further based, at least in part, that low amounts of proteinase K in the presence of a detergent are sufficient to make substantially all AAV genomes in a sample accessible for polymerase chain reaction and thereby determination/quantification. Without being bound by this theory, it is assumed that high concentrations of proteinase K interfere with the polymerase chain reaction and that by using substantially reduced, i.e. low, amounts of proteinase K results in an improved polymerase chain reaction by reducing PCR inhibition. Especially the inhibition of the polymerase chain reaction by proteinaceous substances in cell lysates of AAV producing cells can be reduced or even eliminated by the proteinase K incubation in the presence of a detergent.

The invention is further based, at least in part, on the finding, that for the sequential incubation with a nuclease and a protease of crude cell lysate samples the sample shall not be diluted after the nuclease incubation and the total volume of the incubation mixture shall be used in the protease incubation step.

In general, the more pre-treatment steps are required prior to the determination of the copy number of AAV genomes in a ddPCR method the higher is the likelihood of contamination.

### Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “AAV helper functions” denotes AAV-derived coding sequences (proteins) which can be expressed to provide AAV gene products and AAV particles that, in turn, function in trans for productive AAV replication and packaging. Thus, AAV helper functions include AAV open reading frames (ORFs), including rep and cap and others such as AAP for certain AAV serotypes. The rep gene expression products have been shown to possess many functions, including, among others: recognition, binding and nicking of the AAV origin of DNA replication; DNA helicase activity; and modulation of transcription from AAV (or other heterologous) promoters. The cap gene expression products (capsids) supply necessary packaging functions. AAV helper functions are used to complement AAV functions in trans that are missing from AAV vector genomes.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “comprising” also encompasses the term “consisting of”.

The terms “empty capsid” and “empty particle”, refer to an AAV particle that has an AAV protein shell but that lacks in whole or part a nucleic acid that encodes a protein or is transcribed into a transcript of interest flanked by AAV ITRs, i.e. a vector. Accordingly, the empty capsid does not function to transfer a nucleic acid that encodes a protein or is transcribed into a transcript of interest into the host cell.

The term “endogenous” denotes that something is naturally occurring within a cell; naturally produced by a cell; likewise, an “endogenous gene locus/cell-endogenous gene locus” is a naturally occurring locus in a cell.

As used herein, the term “exogenous” indicates that a nucleotide sequence does not originate from a specific cell and is introduced into said cell by DNA delivery methods, e.g., by transfection, electroporation, or transduction by viral vectors. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the sequence is becoming an “exogenous” sequence by its introduction into the cell, e.g., via recombinant DNA technology.

An “isolated” composition is one, which has been separated from one or more component(s) of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from one or more component(s) of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from one or more component(s) of its natural environment.

The term “mammalian cell comprising an exogenous nucleotide sequence” encompasses cells into which one or more exogenous nucleic acid(s) have been introduced, including the progeny of such cells. These can be the starting point for further genetic modification. Thus, the term “a mammalian cell comprising an exogenous nucleotide sequence” encompasses a cell comprising an exogenous nucleotide sequence integrated at a single site within a locus of the genome of said mammalian cell, wherein the exogenous nucleotide sequence comprises at least a first and a second recombination recognition site (these recombination recognition sites are different) flanking at least one first selection marker. In certain embodiments, the mammalian cell comprising an exogenous nucleotide sequence is a cell comprising an exogenous nucleotide sequence integrated at a single site within a locus of the genome of said cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

A “mammalian cell comprising an exogenous nucleotide sequence” and a “recombinant cell” are both “transfected cells”. This term includes the primary transfected cell as well as progeny derived therefrom without regard to the number of passages. Progeny may, e.g., not be completely identical in nucleic acid content to a parent cell, but may contain mutations. Mutant progeny that has the same function or biological activity as in the originally transfected cell are encompassed.

The “nucleic acids encoding AAV packaging proteins” refer generally to one or more nucleic acid molecule(s) that includes nucleotide sequences providing AAV functions deleted from an AAV vector, which is (are) to be used to produce a transduction competent recombinant AAV particle. The nucleic acids encoding AAV packaging proteins are commonly used to provide expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or particle. A number of nucleic acid constructs have been described, such as the commonly used plasmids pAAV/Ad and pIM29+45, which encode both rep and cap gene expression products. See, e.g., Samulski et al., J. Virol. 63 (1989) 3822-3828; and McCarty et al., J. Virol. 65 (1991) 2936-2945. A number of plasmids have been described which encode rep and/or cap gene expression products (e.g., U.S. Pat. Nos. 5,139,941 and 6,376,237). Any one of these nucleic acids encoding AAV packaging proteins can comprise the DNA element or nucleic acid according to the invention.

The term “nucleic acids encoding helper proteins” refers generally to one or more nucleic acid molecule(s) that include nucleotide sequences encoding proteins and/or RNA molecules that provide adenoviral helper function(s). A plasmid with nucleic acid(s) encoding helper protein(s) can be transfected into a suitable cell, wherein the plasmid is then capable of supporting AAV particle production in said cell. Any one of these nucleic acids encoding helper proteins can comprise the DNA element or nucleic acid according to the invention. Expressly excluded from the term are infectious viral particles, as they exist in nature, such as adenovirus, herpesvirus or vaccinia virus particles.

As used herein, the term “operably linked” refers to a juxtaposition of two or more components, wherein the components are in a relationship permitting them to function in their intended manner. For example, a promoter and/or an enhancer is operably linked to a coding sequence/open reading frame/gene if the promoter and/or enhancer acts to modulate the transcription of the coding sequence/open reading frame/gene. In certain embodiments, DNA sequences that are “operably linked” are contiguous. In certain embodiments, e.g., when it is necessary to join two protein encoding regions, such as a secretory leader and a polypeptide, the sequences are contiguous and in the same reading frame. In certain embodiments, an operably linked promoter is located upstream of the coding sequence/open reading frame/gene and can be adjacent to it. In certain embodiments, e.g., with respect to enhancer sequences modulating the expression of a coding sequence/open reading frame/gene, the two components can be operably linked although not adjacent. An enhancer is operably linked to a coding sequence/open reading frame/gene if the enhancer increases transcription of the coding sequence/open reading frame/gene. Operably linked enhancers can be located upstream, within, or downstream of coding sequences/open reading frames/genes and can be located at a considerable distance from the promoter of the coding sequence/open reading frame/gene.

The term “packaging proteins” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-I) and vaccinia virus.

As used herein, “AAV packaging proteins” refer to AAV-derived sequences, which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

A “plasmid” is a form of nucleic acid or polynucleotide that typically has additional elements for expression (e.g., transcription, replication, etc.) or propagation (replication) of the plasmid. A plasmid as used herein also can be used to reference such nucleic acid or polynucleotide sequences. Accordingly, in all aspects the inventive compositions and methods are applicable to nucleic acids, polynucleotides, as well as plasmids, e.g., for producing cells that produce viral (e.g., AAV) vectors, to produce viral (e.g., AAV) particles, to produce cell culture medium that comprises viral (e.g., AAV) particles, etc.

The term “recombinant cell” as used herein denotes a cell after final genetic modification, such as, e.g., a cell expressing a polypeptide of interest or producing a rAAV particle of interest and that can be used for the production of said polypeptide of interest or rAAV particle of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

A “recombinant AAV vector” is derived from the wild-type genome of a virus, such as AAV by using molecular biological methods to remove the wild type genome from the virus (e.g., AAV), and replacing it with a non-native nucleic acid, such as a nucleic acid transcribed into a transcript or that encodes a protein. Typically, for AAV one or both inverted terminal repeat (ITR) sequences of the wild-type AAV genome are retained in the recombinant AAV vector. A “recombinant” AAV vector is distinguished from a wild-type viral AAV genome, since all or a part of the viral genome has been replaced with a non-native (i.e., heterologous) sequence with respect to the viral genomic nucleic acid. Incorporation of a non-native sequence therefore defines the viral vector (e.g., AAV) as a “recombinant” vector, which in the case of AAV can be referred to as a “rAAV vector.”

A recombinant vector (e.g., AAV) sequence can be packaged-referred to herein as a “particle”—for subsequent infection (transduction) of a cell, ex vivo, in vitro or in vivo. Where a recombinant vector sequence is encapsulated or packaged into an AAV particle, the particle can also be referred to as a “rAAV”. Such particles include proteins that encapsulate or package the vector genome. Particular examples include viral envelope proteins, and in the case of AAV, capsid proteins, such as AAV VP1, VP2 and VP3.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “serotype” is a distinction based on AAV capsids being serologically distinct. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

The terms “transduce” and “transfect” refer to introduction of a molecule such as a nucleic acid (viral vector, plasmid) into a cell. A cell has been “transduced” or “transfected” when exogenous nucleic acid has been introduced inside the cell membrane. Accordingly, a “transduced cell” is a cell into which a “nucleic acid” or “polynucleotide” has been introduced, or a progeny thereof in which an exogenous nucleic acid has been introduced. In particular embodiments, a “transduced” cell (e.g., in a mammal, such as a cell or tissue or organ cell) has a genetic change following incorporation of an exogenous molecule, for example, a nucleic acid (e.g., a transgene). A “transduced” cell(s) can be propagated and the introduced nucleic acid transcribed and/or protein expressed.

In a “transduced” or “transfected” cell, the nucleic acid (viral vector, plasmid) may or may not be integrated into genomic nucleic acid. If an introduced nucleic acid becomes integrated into the nucleic acid (genomic DNA) of the recipient cell or organism, it can be stably maintained in that cell or organism and further passed on to or inherited by progeny cells or organisms of the recipient cell or organism. Finally, the introduced nucleic acid may exist in the recipient cell or host organism extrachromosomally, or only transiently. A number of techniques are known, see, e.g., Graham et al., Virology 52 (1973) 456; Sambrook et al. (1989) Molecular Cloning, a laboratory manual, Cold Spring Harbor Laboratories, New York; Davis et al. (1986) Basic Methods in Molecular Biology, Elsevier; and Chu et al., Gene 13 (1981) 197. Such techniques can be used to introduce one or more exogenous DNA moieties into suitable host cells.

The term “transgene” is used herein to conveniently refer to a nucleic acid that is intended or has been introduced into a cell or organism. Transgenes include any nucleic acid, such as a gene that is transcribed into a transcript or that encodes a polypeptide or protein.

A “vector” refers to the portion of the recombinant plasmid sequence ultimately packaged or encapsulated, either directly or in form of a single strand or RNA, to form a viral (e.g., AAV) particle. In cases recombinant plasmids are used to construct or manufacture recombinant viral particles, the viral particle does not include the portion of the “plasmid” that does not correspond to the vector sequence of the recombinant plasmid. This non-vector portion of the recombinant plasmid is referred to as the “plasmid backbone”, which is important for cloning and amplification of the plasmid, a process that is needed for propagation and recombinant virus production, but is not itself packaged or encapsulated into virus (e.g., AAV) particles. Thus, a “vector” refers to the nucleic acid that is packaged or encapsulated by a virus particle (e.g., AAV).

### Recombinant Cell

Generally, for efficient as well as large-scale production of a proteinaceous compound of interest, such as e.g. a rAAV particle or a therapeutic polypeptide, a cell expressing and, if possible, also secreting said proteinaceous compound is required. Such a cell is termed “recombinant cell” or “recombinant production cell”.

For the generation of a “recombinant production cell” a suitable mammalian cell is transfected with the required nucleic acid sequences encoding said proteinaceous compound of interest. Transfection of additional helper polypeptides may be necessary.

For the generation of stable recombinant production cells, a second step follows, wherein a single cell stably expressing the proteinaceous compound of interest is selected. This can be done, e.g., based on the co-expression of a selection marker, which had been co-transfected with the nucleic acid sequences encoding the proteinaceous compound of interest, or be the expression of the proteinaceous compound itself.

For expression of a coding sequence, i.e. of an open reading frame, additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case the proteinaceous compound of interest is an AAV particle, which is composed of different (monomeric) capsid polypeptides and a single stranded DNA molecule and which in addition requires other adenoviral helper functions for production and encapsulation, a multitude of expression cassettes differing in the contained open reading frames/coding sequences are required. In this case, at least an expression cassette for each of the transgene, the different polypeptides forming the capsid of the AAV vector, for the required helper functions as well as the VA RNA are required. Thus, individual expression cassettes for each of the helper E1A, E1B, E2A, E4orf6, the VA RNA, the rep and cap genes are required.

As outlined in the previous paragraphs, the more complex the proteinaceous compound of interest or the higher the number of additional required helper polypeptides and/or RNAs, respectively, the higher is the number of required, different expression cassettes. Inherently with the number of expression cassettes, also the total size of the nucleic acid. However, there is a practical upper limit to the size of a nucleic acid that can be transferred, which is in the range of about 15 kbps (kilo-base-pairs). Above this limit handling and processing efficiency profoundly drops. This issue can be addressed by using two or more separate plasmids. Thereby the different expression cassettes are allocated to different plasmids, whereby each plasmid comprises only some of the expression cassettes.

In certain embodiments of all aspects and embodiments, each of the expression cassettes comprise in 5′-to-3′ direction a promoter, an open reading frame/coding sequence or an RNA gene and a polyadenylation signal sequence, and/or a terminator sequence. In certain embodiments, the open reading frame encodes a polypeptide and the expression cassette comprises a polyadenylation signal sequence with or without additional terminator sequence. In certain embodiments, the expression cassette comprises a RNA gene, the promoter is a type 2 Pol III promoter and a polyadenylation signal sequence or a polyU terminator is present. See, e.g., Song et al. Biochemical and Biophysical Research Communications 323 (2004) 573-578. In certain embodiments, the expression cassette comprises a RNA gene, the promoter is a type 2 Pol III promoter and a polyU terminator sequence.

In certain embodiments of all aspects and embodiments, the open reading frame encodes a polypeptide, the promoter is the human CMV promoter with or without intron A, the polyadenylation signal sequence is the bGH (bovine growth hormone) polyA signal sequence and the terminator is the hGT (human gastrin terminator).

In certain embodiments of all aspects and embodiments the promoter is the human CMV promoter with intron A, the polyadenylation signal sequence is the bGH polyadenylation signal sequence and the terminator is the hGT, except for the expression cassette of the RNA gene and the expression cassette of the selection marker, wherein for the selection marker the promoter is the SV40 promoter and the polyadenylation signal sequence is the SV40 polyadenylation signal sequence and a terminator is absent, and wherein for the RNA gene the promoter is a wild-type type 2 polymerase III promoter and the terminator is a polymerase II or III terminator.

### Adeno-Associated Virus (Aav)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An adeno-associated virus (AAV) is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 10 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAVrh-10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The AAV genome harbors three transcription promoters P5, P19, and P40 (Laughlin et al., Proc. Natl. Acad. Sci. USA 76 (1979) 5567-5571) for the expression of the rep and cap genes. The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter in an AAV vector (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products are required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny, single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus.

VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV viral particles containing a DNA molecule are infectious. Inside the infected cell, the parental infecting single strand is converted into a double strand, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Viral vectors such as parvo-virus particles, including AAV serotypes and variants thereof, provide a means for delivery of nucleic acid into cells ex vivo, in vitro and in vivo, which encode proteins such that the cells express the encoded protein. AAVs are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

Viral vectors, which may be used, include, but are not limited to, adeno-associated virus (AAV) particles of multiple serotypes (e.g., AAV-1 to AAV-12, and others) and hybrid/chimeric AAV particles.

AAV particles may be used to advantage as vehicles for effective gene delivery. Such particles possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such vectors typically have one or more of the wild-type AAV genes deleted in whole or in part, for example, rep and/or cap genes, but retain at least one functional flanking ITR sequence, as necessary for the rescue, replication, and packaging of the recombinant vector into an AAV particle. For example, only the essential parts of the vector e.g., the ITR and LTR elements, respectively, are included. An AAV vector genome would therefore include sequences required in cis for replication and packaging (e.g., functional ITR sequences).

Recombinant AAV vectors, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, 218, AAV rh74 or AAV 7m8 for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be an AAV1, AAV3, AAV4, AAV5, AAV6, AAV7, AAV8, AAV9, AAV10, AAV11, AAV12, AAV-218, AAV rh74, AAV 7m8 or a variant thereof, for example. AAV variants include variants and chimeras of AAV1, AAV2, AAV3, AAV4, AAV5, AAV6, AAV7, AAV8, AAV9, AAV10, AAV11, AAV12, AAV-218, AAV rh74 and AAV 7m8 capsids.

In certain embodiments of all aspects and embodiments, adeno-associated virus (AAV) vectors include AAV1, AAV2, AAV3, AAV4, AAV5, AAV6, AAV7, AAV8, AAV9, AAV10, AAV11, AAV12, AAV-218, AAV rh74 and AAV 7m8, as well as variants (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

AAV and AAV variants (e.g., capsid variants) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

In certain embodiments of all aspects and embodiments, an AAV particle related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, AAV-218, AAV rh74 or AAV 7m8 (e.g., such as an ITR, or a VP1, VP2, and/or VP3 sequences).

Compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, AAV-218, AAV rh74, or AAV 7m8, but are distinct from and not identical to known AAV genes or proteins, such as AAV1, AAV2, AAV3, AAV4, AAV5, AAV6, AAV7, AAV8, AAV9, AAV10, AAV11, AAV12, AAV-218, AAV rh74, or AAV 7m8, genes or proteins, etc. In certain embodiments of all aspects and embodiments, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, AAV-218, AAV rh74, or AAV 7m8 (e.g., VP1, VP2 and/or VP3 capsid or ITR). In certain embodiments, an AAV variant has 1, 2, 3, 4, 5, 5-10, 10-15, 15-20 or more amino acid substitutions.

Recombinant AAV particles, including AAV1, AAV2, AAV3, AAV4, AAV5, AAV6, AAV7, AAV8, AAV9, AAV10, AAV11, AAV12, AAV-218, AAV rh74 or AAV 7m8, and variant, related, hybrid and chimeric sequences, can be constructed using recombinant techniques that are known to the skilled artisan, to include one or more nucleic acid sequences (transgenes) flanked with one or more functional AAV ITR sequences.

Recombinant particles (e.g., rAAV particles) can be incorporated into pharmaceutical compositions. Such pharmaceutical compositions are useful for, among other things, administration and delivery to a subject in vivo or ex vivo. In certain embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

### SPECIFIC EMBODIMENTS OF THE METHOD ACCORDING TO THE INVENTION

In order to allow viral genome copy determination, the viral genomes have to be made accessible, i.e. the shielding capsid must be opened. For this, heat denaturation is convenient and commonly used. However, it has been found that heat denaturation results in artificially lowered viral genome copy numbers.

The current inventors have shown that heat denaturation at temperatures above 95° C., such as e.g. 98° C., results in a reduction of the determined viral copy genome number. This has been exemplified using the ATCC AAV2 standard VR-1616. The lot used in the experiments has a nominal viral genome copy number (vgcn) of 3.28*10E10 vg/mL. The results are shown in the following Table 1.

TABLE 1

| condition | absolute vgcn | relative vgcn |
|---|---|---|
| no pre-treatment | 2.735-3.306*10E10/mL | 83-101% |
| heat denaturation @ 98° | 1.012*10E10/mL | 31% |
| C., 10 min. | | |

Likewise, the current inventors have shown that incubation with proteinase K (PK) in aqueous or buffered solution in the absence of a detergent also results in a reduction of the determined viral copy genome number. This has been exemplified using the ATCC AAV2 standard VR-1616. The lot used in the experiments has a nominal viral genome copy number (vgcn) of 3.28*10E10 vg/mL. The results are shown in the following Table 2.

TABLE 2

| condition | absolute vgcn | relative vgcn |
|---|---|---|
| no pre-treatment | 2.735-3.306*10E10/mL | 83-101% |
| 2 mU PK in water, 50° C., | 1.202*10E9/mL | 4% |
| 60 min.; 95° C., 15 min.; | | |
| 4° C. | | |
| 2 mU PK in 20 mM Tris- | 1.327*10E10/mL | 40% |
| HCl, 1 mM EDTA, 100 mM | | |
| NaCl, 50° C., 60 min.; | | |
| 95° C., 15 min.; 4° C. | | |

In combination, the current inventors have shown that heat denaturation at temperatures above 95° C., such as e.g. 98° C., even in combination with an incubation with proteinase K (PK) in buffer in the absence of a detergent results in a reduction of the determined viral copy genome number. This has been exemplified using the ATCC AAV2 standard VR-1616. The lot used in the experiments has a nominal viral genome copy number (vgcn) of 3.28*10E10 vg/mL. The results are shown in the following Table 3.

TABLE 3

| condition | absolute vgcn | relative vgcn |
|---|---|---|
| no pre-treatment | 2.735-3.306*10E10/mL | 83-101% |
| heat denaturation @ 98° | 1.814*10E10/mL | 55% |
| C., 10 min.; 1 mU PK in | | |
| 20 mM Tris-HCl, 1 mM | | |
| EDTA, 100 mM NaCl, | | |
| 50° C., 60 min.; 95° C., | | |
| 15 min.; 4° C. | | |

The current inventors have now found that the reduction of the determined viral genome copy genome number can be overcome by performing the incubation with proteinase K in the presence of sodium dodecyl sulfate (SDS). This is shown in this example using the ATCC AAV2 standard VR-1616. The lot used in the experiments has a nominal viral genome copy number (vgcn) of 3.28*10E10 vg/mL. The results are shown in the following Table 4.

TABLE 4

| condition | absolute vgcn | relative vgcn |
|---|---|---|
| no pre-treatment | 2.735-3.306*10E10/mL | 83-101% |
| 2 mU PK in 20 mM Tris- | 3.520*10E10/mL | 107% |
| HCl, 1 mM EDTA, 100 | | |
| mM NaCl, 1% SDS, 50° | | |
| C., 60 min.; 95° C., | | |
| 15 min.; 4° C. | | |

The recovery values obtained with the conditions of Table 4 are fulfilling the acceptance criteria for the validation of assay procedures according to EMA and FDA guidelines, i.e. are within +/−15% of the nominal value.

The current inventors have further shown that treatment with DNase I prior to viral genome number determination results in a reduction of the determined viral copy genome number. This has been exemplified using the ATCC AAV2 standard VR-1616. The lot used in the experiments has a nominal viral genome copy number (vgcn) of 3.28*10E10 vg/mL. The results are shown in the following Table 5.

TABLE 5

| condition | absolute vgcn | relative vgcn |
|---|---|---|
| no pre-treatment | 2.735-3.306*10E10/mL | 83-101% |
| 5 U DNase I, 40 mM | 1.795*10E10/mL | 55% |
| Tris-HCl, pH 8.0, 10 mM | | |
| MgSO4, 1 mM CaCl2, | | |
| 37° C., 30 min.; 95° C., | | |
| 15 min. | | |

The reduction by the DNase I treatment is independent from the further processing of the sample, i.e. of heat denaturation or proteinase K incubation. The different tested conditions, which all show a reduction of the determined viral copy number, are summarized in Table 6. The ATCC AAV2 standard VR-1616 has been used. The lot used in the experiments has a nominal viral genome copy number (vgcn) of 3.28*10E10 vg/mL.

TABLE 6

| condition | absolute vgcn | relative vgcn |
|---|---|---|
| no pre-treatment | 2.735-3.306*10E10/mL | 83-101% |
| 5 U DNase I, 40 mM Tris-HCl, pH 8.0, 10 mM MgSO4, | 1.795*10E10/mL | 55% |
| 1 mM CaCl2, 37° C., 30 min.; 95° C., 15 min. | | |
| 5 U DNase I, 40 mM Tris-HCl, pH 8.0, 10 mM | 4.947*10E9/mL | 15% |
| MgSO4, 1 mM CaCl2, 37° C., 30 min.; 95° C., | | |
| 15 min.; 2 mU PK in water, 50° C., 60 min.; | | |
| 95° C., 15 min.; 4° C. | | |
| 5 U DNase I, 40 mM Tris-HCl, pH 8.0, 10 mM | 9.590*10E9/mL | 29% |
| MgSO4, 1 mM CaCl2, 37° C., 30 min.; 95° C., | | |
| 15 min.; 2 mU PK in 20 mM Tris-HCl, 1 mM EDTA, 100 | | |
| mM NaCl, 50° C., 60 min.; 95° C., 15 min.; 4° C. | | |
| 5 U DNase I, 40 mM Tris-HCl, pH 8.0, 10 mM MgSO4, 1 mM | 2.347*10E10/mL | 72% |
| CaCl2, 37° C., 30 min.; 95° C., 15 min.; 2 mU PK in 20 mM | | |
| Tris-HCl, 1 mM EDTA, 100 mM NaCl, 1% SDS, 50° C., 60 min.; | | |
| 95° C., 15 min.; 4° C. | | |

The finding by the current inventors was confirmed using lysates of an AAV2 producing HEK293 cell cultivation. The established viral genome copy number (vgcn) in the lysate was 1.746*10E10 vg/mL (lysate 18). The results are shown in the following Table 7.

It can be seen that incubation with proteinase K allows for a recovery of 96% of the viral genomes. This can further be increased by incubating the sample with proteinase K in the presence of SDS to 100%.

TABLE 7

| condition | absolute vgcn | relative vgcn |
|---|---|---|
| no pre-treatment | 3.594*10E9/mL | 21% |
| heat denaturation @ 98° | 3.514*10E9/mL | 20% |
| C., 10 min. | | |
| 2 mU PK in water, 50° C., | 7.636*10E9/mL | 44% |
| 60 min.; 95° C., 15 min.; | | |
| 4° C. | | |
| heat denaturation @ 98° | 7.020*10E9/mL | 40% |
| C., 10 min.; 2 mU PK in | | |
| 20 mM Tris-HCl, 1 mM | | |
| EDTA, 100 mM NaCl, 50° | | |
| C., 60 min.; 95° C., 15 | | |
| min.; 4° C. | | |
| 2 mU PK in 20 mM Tris- | 1.671*10E10/mL | 96% |
| HCl, 1 mM EDTA, 100 | | |
| mM NaCl, 50° C., 60 | | |
| min.; 95° C., 15 min.; | | |
| 4° C. | | |
| 2 mU PK in 20 mM Tris- | 1.746*10E10/mL | 100% |
| HCl, 1 mM EDTA, 100 | | |
| mM NaCl, 1% SDS, 50° | | |
| C., 60 min.; 95° C., | | |
| 15 min.; 4° C. | | |

It has to be pointed out that no further purification of the proteinase K incubated lysate had been performed, i.e., for example, no column-based or extractive purification.

The determined viral genome copy number with additional DNase I treatment were in the range of 4% to 16% only as shown in the following Table 8.

TABLE 8

| condition | absolute vgcn | relative vgcn |
|---|---|---|
| no pre-treatment | 3.594*10E9/mL | 21% |
| 5 U DNase I, 40 mM | 6.960*10E8/mL | 4% |
| Tris-HCl, pH 8.0, 10 mM | | |
| MgSO4, 1 mM CaCl2, | | |
| 37° C., 30 min.; 95° C., | | |
| 15 min. | | |
| 5 U DNase I, 40 mM | 1.179*10E9/mL | 7% |
| Tris-HCl, pH 8.0, 10 mM | | |
| MgSO4, 1 mM CaCl2, | | |
| 37° C., 30 min.; 95° C., | | |
| 15 min.; 2 mU PK in water, | | |
| 50° C., 60 min.; 95° C., | | |
| 15 min.; 4° C. | | |
| 5 U DNase I, 40 mM | 1.746*10E9/mL | 10% |
| Tris-HCl, pH 8.0, 10 mM | | |
| MgSO4, 1 mM CaCl2, | | |
| 37° C., 30 min.; 95° C., | | |
| 15 min.; 2 mU PK in 20 | | |
| mM Tris-HCl, 1 mM EDTA, | | |
| 100 mM NaCl, 50° C., 60 | | |
| min.; 95° C., 15 min.; 4° C. | | |
| 5 U DNase I, 40 mM | 2.716*10E9/mL | 16% |
| Tris-HCl, pH 8.0, 10 mM | | |
| MgSO4, 1 mM CaCl2, | | |
| 37° C., 30 min.; 95° C., | | |
| 15 min.; 2 mU PK in 20 mM | | |
| Tris-HCl, 1 mM EDTA, | | |
| 100 mM NaCl, 1% SDS, | | |
| 50° C., 60 min.; 95° C., | | |
| 15 min.; 4° C. | | |

The current inventors have found that for the sequential incubation with DNase I and proteinase K it is advantageous to use the entire incubation mixture of the DNase I incubation for the proteinase K incubation. Thereby the determined viral genome copy number could be increased to 80% of the established number. The established viral genome copy number (vgcn) in the lysate was 1.746*10E10 vg/mL (lysate 18) and 2.340*10E9 vg/mL (lysate 31). Such an effect cannot be seen with purified samples, such as the ATCC standard (nominal viral genome copy number (vgcn) of 3.28*10E10 vg/mL). The results are shown in Table 9.

TABLE 9

| condition | absolute vgcn | relative vgcn |
|---|---|---|
| 10 μL lysate 18 for | 2.716*10E9/mL | 16% |
| DNase I incubation; 1:10 | | |
| diluted for PK incubation | | |
| 10 μL lysate 31 for | 1.872*10E9/mL | 80% |
| DNase I incubation; no | | |
| dilution for PK incubation | | |
| 10 μL ATCC standard | 2.347*10E10/mL | 72% |
| for DNase I incubation; | | |
| 1:10 diluted for PK incubation | | |
| 10 μL ATCC standard | 2.468*10E10/mL | 75% |
| for DNase I incubation; | | |
| no dilution for PK incubation | | |

The current inventors have found that a recovery of more than 80% of viral genomes can be achieved using the combination of DNase I and proteinase K incubation without dilution using affinity chromatography purified cell lysates. The established viral genome copy number (vgcn) in the affinity purified lysate was 5.110*10E10 vg/mL (affinity purified lysate 31) and 7.320*10E10 vg/mL (affinity purified lysate 33). The results are shown in Table 10.

TABLE 10

| condition | absolute vgcn | relative vgcn |
|---|---|---|
| 10 μL affinity purified | 4.430*10E10/mL | 87% |
| lysate 31 for DNase I | | |
| incubation; no dilution | | |
| for PK incubation | | |
| 10 μL affinity purified | 7.320*10E10/mL | 100% |
| lysate 33 for DNase I | | |
| incubation; no dilution | | |
| for PK incubation | | |

VIRAL GENOME QUANTIFICATION

In order to allow correct viral genome quantification the sample must be free of plasmid DNA as well as unpackaged vector genomes, both containing at least parts of the viral genome. Further, the packaged AAV genomes must be available from the first PCR cycle.

To remove unpackaged DNA, which might interfere in the ddPCR process, DNase I digest is commonly used.

To make the encapsulated DNA accessible for ddPCR, capsid opening is required. This can be done either by incubation at high temperature or proteinase K digest. The requirement for proteinase K digestion at all, is heavily discussed in the art.

### Droplet Digital Polymerase Chain Reaction

Droplet digital PCR (ddPCR) allows for the absolute quantification of viral genomes without the need for the generation of a standard curve.

In more detail, droplet digital polymerase chain reaction (ddPCR) enables absolute quantification of nucleic acids by randomly distributing a PCR reaction mixture into discrete partitions, where some have no nucleic target sequences and others have one or more template copies present (Hindson, B., et al., Anal. Chem. 83 (2011) 8604-8610). Due to partitioning, thousands of independent PCR reactions are performed during thermal cycling. At the endpoint, the fraction of target-positive partitions is read out and used for the calculation of initial template DNA concentration (Pinheiro, L., et al., Anal. Chem. 84 (2011) 1003-1011).

First, up to 20,000 droplets with a volume of about 1 nL are formed in a water-oil emulsion. Thereby the PCR reaction mix, comprising of the nucleic acid template, forward (fwd) and reverse (rev) primer, a TaqMan probe and a ddPCR supermix, which contains a Thermus aquaticus (Taq) DNA polymerase, dNTPs and PCR buffer, is partitioned (see, e.g., Hindson, B., et al. 2011; Taylor, S, et al., Sci. Rep. 7 (2017) 2409). In each droplet, an individual PCR reaction is carried out during thermal cycling, depending on presence or absence of the DNA target.

In droplets containing template DNA, target sequences are amplified. During amplification, the 5′-to-3′ exonuclease activity of Taq polymerase hydrolyses the TaqMan probe, which is bound to the template strand. Due to degradation of the probe into smaller fragments, the 5′-located fluorophore is no longer in close proximity to its 3′-located quencher. Thereby signal quenching is abolished and a fluorescence signal is generated. Partitions lacking template sequences show no amplification and therefore no hydrolysis of TaqMan probes and fluorescence generation, respectively, as the fluorescence of the 5′-located fluorophore remains quenched (see, e.g., Holland, P., et al., Proc. Natl. Acad. Sci. USA 88 (1991) 7276-7580). Since probes with distinct fluorophores are available, that have different excitation and emission wavelengths, ddPCR reactions can be performed as multiplex reactions within one droplet. Commonly used fluorophores for two-dimensional ddPCR are 6-carboxyfluorescein (FAM) and hexachloro-6-carboxyfluorescein (HEX), both quenched by black hole quencher 1 (BHQ1) (see, e.g., Furuta-Hanawa, B., et al. Hum. Gen. Therap. Meth. 30 (2019) 127-136).

As an endpoint analysis, the fluorescence signal of each droplet after thermal cycling is read out. Using Poisson statistics, the copy number of target sequences (2) can be calculated from the ratio of positive to total readouts (p), according to equation 1 (see, e.g., Hindson, B., et al. (2011)).

$$λ = - ln ( 1 - p ) \qquad ( 1 )$$

Because ddPCR relies on an endpoint measurement, target sequence quantification is to a certain extent independent of the PCR reaction efficiency. This is in contrast to real time PCR (qPCR), which is commonly used for viral genome titration (see, e.g., Taylor, S, et al. (2017)). Further, no standards or calibration samples need to be used (see, e.g., Dorange, F., Bec, C., Cell Gen. Therap. Ins. 4 (2018) 119-129).

### Recombinant AAV Particles

Different methods that are known in the art for generating rAAV particles. For example, transfection using AAV plasmid and AAV helper sequences in conjunction with co-infection with one AAV helper virus (e.g., adenovirus, herpesvirus, or vaccinia virus) or transfection with a recombinant AAV plasmid, an AAV helper plasmid, and an helper function plasmid. Non-limiting methods for generating rAAV particles are described, for example, in U.S. Pat. Nos. 6,001,650, 6,004,797, WO 2017/096039, and WO 2018/226887. Following recombinant rAAV particle production (i.e. particle generation in cell culture systems), rAAV particles can be obtained from the host cells and cell culture supernatant and purified.

For the generation of recombinant AAV particles, expression of the Rep and Cap proteins, the helper proteins E1A, E1B, E2A and E4orf6 as well as the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be used.

Generally, to produce recombinant AAV particles, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for AAV replication.

To reduce the number of required plasmids, Rep, Cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

Thus, cell lines in which the rep gene is integrated and expressed tend to grow slowly or express Rep proteins at very low levels.

In order to limit the transgene activity to specific tissues, i.e. to limit the site of integration the transgene can be operably linked to an inducible or tissue specific promoter (see, e.g., Yang, Y., et al. Hum. Gene. Ther. 6 (1995) 1203-1213).

One difficulty in the production of rAAV particles is the inefficient packaging of the rAAV vector, resulting in low titers. Packaging has been difficult for several reasons including
preferred encapsidation of wild-type AAV genomes if they are present;
difficulty in generating sufficient complementing functions such as those provided by the wild-type rep and cap genes due to the inhibitory effect associated with the rep gene products;
the limited efficiency of the co-transfection of the plasmid constructs.

All this is based on the biological properties of the Rep proteins. Especially the inhibitory (cytostatic and cytotoxic) properties of the Rep proteins as well as the ability to reverse the immortalized phenotype of cultured cells is problematic. Additionally, Rep proteins down-regulate their own expression when the widely used AAV P5 promoter is employed (see, e.g., Tratschin et al., Mol. Cell. Biol. 6 (1986) 2884-2894).

In certain embodiments of all aspects and embodiments, the rAAV particles are derived from an AAV selected from the group consisting of AAV1, AAV2, AAV3, AAV4, AAV5, AAV6, AAV7, AAV8, AAV9, AAV10, Rh. 10, Rh74 and 7m8.

In certain embodiments of all aspects and embodiments, the rAAV particles comprise a capsid sequence having 70% or more sequence identity to an AAV1, AAV2, AAV3, AAV4, AAV5, AAV6, AAV7, AAV8, AAV9, AAV10, Rh. 10, Rh74, or 7m8 capsid sequence.

In certain embodiments of all aspects and embodiments, the rAAV particles comprise an ITR sequence having 70% or more sequence identity to an AAV1, AAV2, AAV3, AAV4, AAV5, AAV6, AAV7, AAV8, AAV9, or AAV10 ITR sequence.

### E1A, E1B, E2 and E4

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK 146 as reported in EP 1 230 354, as well as plasmids pGS119 and pGS122 as reported in WO 2007/056994, can also be used a source for the E1A and E1B open reading frames.

E1A is the first viral helper gene that is expressed after adenoviral DNA enters the cell nucleus. The E1A gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will dye (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

Also the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

### Adenoviral VA RNA Gen

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289) from a type 2 polymerases III promoter. For recombinant production the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

Methods for Producing rAAV Particles

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Aspects of the current invention are methods of transducing cells with nucleic acids (e.g., plasmids) comprising all required elements for the production of recombinant AAV particles, wherein the viral genome copy number is determined with a method according to the current invention. Thus, as the plasmids encode viral packaging proteins and/or helper proteins the cells can produce recombinant viral particles that include a nucleic acid that encodes a protein of interest or comprises a sequence that is transcribed into a transcript of interest.

The invention provides a viral (e.g., AAV) particle production platform that includes features that distinguish it from current ‘industry-standard’ viral (e.g., AAV) particle production processes by using the method according to the current invention.

More generally, cells transfected or transduced with DNA for the recombinant production of AAV particles can be referred to as “recombinant cell”. Such a cell can be, for example, a yeast cell, an insect cell, or a mammalian cell, and has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

Helper protein plasmids can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. Ito et al., J. Gen. Virol. 9 (1970) 243; Ishibashi et al., Virology 45 (1971) 317.

Mutants within the E2B and E3 regions have been shown to support AAV replication, indicating that the E2B and E3 regions are probably not involved in providing helper function. Carter et al., Virology 126 (1983) 505. However, adenoviruses defective in the E1 region, or having a deleted E4 region, are unable to support AAV replication. Thus, for adenoviral helper proteins, E1A and E4 regions are likely required for AAV replication, either directly or indirectly (see, e.g., Laughlin et al., J. Virol. 41 (1982) 868; Janik et al., Proc. Natl. Acad. Sci. USA 78 (1981) 1925; Carter et al., Virology 126 (1983) 505). Other characterized adenoviral mutants include: E1B (Laughlin et al. (1982), supra; Janik et al. (1981), supra; Ostrove et al., Virology 104 (1980) 502); E2A (Handa et al., J. Gen. Virol. 29 (1975) 239; Strauss et al., J. Virol. 17 (1976) 140; Myers et al., J. Virol. 35 (1980) 665; Jay et al., Proc. Natl. Acad. Sci. USA 78 (1981) 2927; Myers et al., J. Biol. Chem. 256 (1981) 567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the E1B have reported that the E1B 55 kDa protein is required for AAV particle production, while E1B 19 kDa is not. In addition, WO 97/17458 and Matshushita et al. (Gene Therapy 5 (1998) 938-945) described helper function plasmids encoding various adenoviral genes. An example of a helper plasmid comprise an adenovirus VA RNA coding region, an adenovirus E4orf6 coding region, an adenovirus E2A 72 kDa coding region, an adenovirus E1A coding region, and an adenovirus

E1B region lacking an intact E1B 55 kDa coding region (see, e.g., WO 01/83797).

Thus, herein is provided a method for producing recombinant AAV vectors or AAV particles comprising said recombinant AAV vectors, which comprise a nucleic acid that encodes a protein or is transcribed into a transcript of interest, using the method according to the current invention for viral genome number determination.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii) contacting one or more mammalian or insect cells with the provided plasmids;
(iv) either further adding a transfection reagent and optionally incubating the plasmid/transfection reagent/cell mixture; or providing a physical means, such as an electric current, to introduce the nucleic acid into the cells;
(v) cultivating the transfected cells;
(vi) harvesting the cultivated cells and/or culture medium from the cultivated cells to produce a cell and/or culture medium harvest; and
(vii) lysing the cells and optionally isolating recombinant AAV vector or AAV particle from the cell and/or culture medium harvest lysate;
(viii) determining the viral genome copy number in or after steps (vi) or/and step (vii) with a method according to the current invention;
thereby producing a recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii)
   (a) either generating a stable transfected cell by
      contacting one or more mammalian or insect cells with the provided plasmids of (i);
      either further adding a transfection reagent and optionally incubating the plasmid/transfection reagent/cell mixture; or providing a physical means, such as an electric current, to introduce the nucleic acid into the cells;
      selecting a first stably transfected cell;
      contacting the selected first stably transfected cell with the provided plasmid of (ii); and
      either further adding a transfection reagent and optionally incubating the plasmid/transfection reagent/cell mixture; or providing a physical means, such as an electric current, to introduce the nucleic acid into the cells;
   (b) or generating a transient transfected cell by
      contacting one or more mammalian or insect cells with the provided plasmids of (i) and (ii); and
      either further adding a transfection reagent and optionally incubating the plasmid/transfection reagent/cell mixture; or providing a physical means, such as an electric current, to introduce the nucleic acid into the cells;
(iv) cultivating the transfected cell of (iii);
(v) harvesting the cultivated cells and/or culture medium from the cultivated cells to produce a cell and/or culture medium harvest;
(vi) lysing the cells and optionally isolating recombinant AAV vector or AAV particle from the cell and/or culture medium harvest lysate;
(vii) determining the viral genome copy number in or after step (v) or/and step (vi) with the method according to the current invention;
thereby producing recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing a mammalian or insect cell comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii)
   (a) either generating a stable transfected cell by
      contacting one or more mammalian or insect cells with the provided plasmids of (i);
      either further adding a transfection reagent and optionally incubating the plasmid/transfection reagent/cell mixture; or providing a physical means, such as an electric current, to introduce the nucleic acid into the cells;
      selecting a first stably transfected cell;
      contacting the selected first stably transfected cell with the provided plasmid of (ii); and
      either further adding a transfection reagent and optionally incubating the plasmid/transfection reagent/cell mixture; or providing a physical means, such as an electric current, to introduce the nucleic acid into the cells;
   (b) or generating a transient transfected cell by
      contacting one or more mammalian or insect cells with the provided plasmids of (i) and (ii); and
      either further adding a transfection reagent and optionally incubating the plasmid/transfection reagent/cell mixture; or providing a physical means, such as an electric current, to introduce the nucleic acid into the cells;
(iv) cultivating the transfected cell of (iii);
(v) harvesting the cultivated cells and/or culture medium from the cultivated cells to produce a cell and/or culture medium harvest;
(vi) lysing the cells and optionally isolating and/or purifying recombinant AAV vector or AAV particle from the cell and/or culture medium harvest; and
(vii) determining the viral genome copy number in or after step (v) or/and step (vi) with the method according to the current invention;
thereby producing recombinant AAV vector or AAV particle comprising a nucleic acid that encodes a protein of interest or is transcribed into a transcript of interest.

The introduction of the nucleic acid (plasmids) into cells can be done in multiple ways.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred. In certain embodiments of all aspects and embodiments, the nucleic acid (plasmid) is provided in a composition in combination with polyethylenimine (PEI), optionally in combination with cells. In certain embodiments, the composition includes a plasmid/PEI mixture, which has a plurality of components: (a) one or more plasmids comprising nucleic acids encoding AAV packaging proteins and/or nucleic acids encoding helper proteins; (b) a plasmid comprising a nucleic acid that encodes a protein or is transcribed into a transcript of interest; and (c) a polyethylenimine (PEI) solution. In certain embodiments, the plasmids are in a molar ratio range of about 1:0.01 to about 1:100, or are in a molar ratio range of about 100:1 to about 1:0.01, and the mixture of components (a), (b) and (c) has optionally been incubated for a period of time from about 10 seconds to about 4 hours.

In certain embodiments of all aspects and embodiments, the compositions further comprise cells. In certain embodiments, the cells are in contact with the plasmid/PEI mixture of components (a), (b) and/or (c).

In certain embodiments of all aspects and embodiments, the composition, optionally in combination with cells, further comprise free PEI. In certain embodiments, the cells are in contact with the free PEI.

In certain embodiments of all aspects and embodiments, the cells have been in contact with the mixture of components (a), (b) and/or (c) for at least about 4 hours, or about 4 hours to about 140 hours, or for about 4 hours to about 96 hours. In one preferred embodiment, the cells have been in contact with the mixture of components (a), (b) and/or (c) and optionally free PEI, for at least about 4 hours.

The composition may comprise further plasmids or/and cells. Such plasmids and cells may be in contact with free PEI. In certain embodiments, the plasmids and/or cells have been in contact with the free PEI for at least about 4 hours, or about 4 hours to about 140 hours, or for about 4 hours to about 96 hours.

The invention also provides methods for producing transfected cells. The method includes the steps of providing one or more plasmids; providing a solution comprising polyethylenimine (PEI); and mixing the plasmid(s) with the PEI solution to produce a plasmid/PEI mixture. In certain embodiments, such mixtures are incubated for a period in the range of about 10 seconds to about 4 hours. In such methods, cells are then contacted with the plasmid/PEI mixture to produce a plasmid/PEI cell culture; then free PEI is added to the plasmid/PEI cell culture produced to produce a free PEI/plasmid/PEI cell culture; and then the free PEI/plasmid/PEI cell culture produced is incubated for at least about 4 hours, thereby producing transfected cells. In certain embodiments, the plasmids comprise one or more or all of a rep open reading frame, a cap open reading frame, E1A, E1B, E2 and E4orf6 open reading frames and a nucleic acid that encodes a protein or is transcribed into a transcript of interest.

Further provided are methods for producing transfected cells that produce recombinant AAV vector or AAV particle, which include providing one or more plasmids comprising nucleic acids encoding AAV packaging proteins and/or nucleic acids encoding helper proteins; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period in the range of about 10 seconds to about 4 hours); contacting cells with the plasmid/PEI mixture, to produce a plasmid/PEI cell culture; adding free PEI to the plasmid/PEI cell culture produced to produce a free PEI/plasmid/PEI cell culture; and incubating the free PEI/plasmid/PEI cell culture for at least about 4 hours, thereby producing transfected cells that produce recombinant AAV vector or particle comprising a nucleic acid that encodes a protein or is transcribed into a transcript of interest, whereby the viral genome copy number is determined with a method according to the current invention.

Additionally provided are methods for producing recombinant AAV vector or AAV particle comprising a nucleic acid that encodes a protein or is transcribed into a transcript of interest, which includes providing one or more plasmids comprising nucleic acids encoding AAV packaging proteins and/or nucleic acids encoding helper proteins; providing a plasmid comprising a nucleic acid that encodes a protein of interest or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time in the range of about 10 seconds to about 4 hours); contacting cells with the plasmid/PEI mixture produced as described to produce a plasmid/PEI cell culture; adding free PEI to the plasmid/PEI cell culture produced as described to produce a free PEI/plasmid/PEI cell culture; incubating the plasmid/PEI cell culture or the free PEI/plasmid/PEI cell culture produced for at least about 4 hours to produce transfected cells; harvesting the transfected cells produced and/or culture medium from the transfected cells produced to produce a cell and/or culture medium harvest; lysing the cells and optionally isolating the recombinant AAV vector or particle from the cell and/or culture medium harvest lysate, whereby the viral genome copy number is determined in the lysate or the isolated AAV particle with a method according to the current invention; and thereby producing recombinant AAV vector or particle comprising a nucleic acid that encodes a protein or is transcribed into a transcript of interest.

Methods for producing recombinant AAV vectors or AAV particles using the method according to the current invention can include one or more further steps or features. An exemplary step or feature includes, but is not limited to, a step of harvesting the cultivated cells produced and/or harvesting the culture medium from the cultivated cells produced to produce a cell and/or culture medium harvest. An additional exemplary step or feature includes, but is not limited to lysing the harvested cells and optionally isolating the recombinant AAV vector or AAV particle from the cell and/or culture medium harvest lysate; whereby the viral genome copy number is determined using a method according to the current invention; and thereby producing recombinant AAV vector or AAV particle comprising a nucleic acid that encodes a protein or is transcribed into a transcript of interest.

In certain embodiments of all aspects and embodiments, PEI is added to the plasmids and/or cells at various time points. In certain embodiments, free PEI is added to the cells before, at the same time as, or after the plasmid/PEI mixture is contacted with the cells.

In certain embodiments of all aspects and embodiments, the cells are at particular densities and/or cell growth phases and/or viability when contacted with the plasmid/PEI mixture and/or when contacted with the free PEI. In one preferred embodiment, cells are at a density in the range of about 1×10E5 cells/mL to about 1×10E8 cells/mL when contacted with the plasmid/PEI mixture and/or when contacted with the free PEI. In certain embodiments, viability of the cells when contacted with the plasmid/PEI mixture or with the free PEI is about 60% or greater than 60%, or wherein the cells are in log phase growth when contacted with the plasmid/PEI mixture, or viability of the cells when contacted with the plasmid/PEI mixture or with the free PEI is about 90% or greater than 90%, or wherein the cells are in log phase growth when contacted with the plasmid/PEI mixture or with the free PEI.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E1A and E1B, adenovirus E2 and/or E4, VA RNA, and/or non-AAV helper proteins.

In certain embodiments of all aspects and embodiments, the nucleic acids (plasmids) are used at particular amounts or ratios. In certain embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 μg to about 15 μg per mL of cells. In certain embodiments, the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins is in the range of about 1:5 to about 1:1, or is in the range of about 1:1 to about 5:1.

In certain embodiments of all aspects and embodiments, a first plasmid comprises the nucleic acids encoding AAV packaging proteins and a second plasmid comprises the nucleic acids encoding helper proteins.

In certain embodiments of all aspects and embodiments, the molar ratio of the plasmid comprising the nucleic acid that encodes a protein or is transcribed into a transcript of interest to a first plasmid comprising the nucleic acids encoding AAV packaging proteins to a second plasmid comprising the nucleic acids encoding helper proteins is in the range of about 1-5:1:1, or 1:1-5:1, or 1:1:1-5 in co-transfection.

In certain embodiments of all aspects and embodiments, the cell is a eukaryotic cell. In certain embodiments, the eukaryotic cell is a mammalian cell. In one preferred embodiment, the cell is a HEK293 cell or a CHO cell.

The cultivation can be performed using the generally used conditions for the cultivation of eukaryotic cells of about 37° C., 95% humidity and 8 vol.-% CO₂. The cultivation can be performed in serum containing or serum free medium, in adherent culture or in suspension culture. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

Methods according to the current invention include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Methods according to the invention that provide or include AAV vectors or particles can also include other elements. Examples of such elements include but are not limited to: an intron, an expression control element, one or more adeno-associated virus (AAV) inverted terminal repeats (ITRs) and/or a filler/stuffer polynucleotide sequence. Such elements can be within or flank the nucleic acid that encodes a protein or is transcribed into a transcript of interest, or the expression control element can be operably linked to nucleic acid that encodes a protein or is transcribed into a transcript of interest, or the AAV ITR(s) can flank the 5′- or 3′-terminus of nucleic acid that encodes a protein or is transcribed into a transcript of interest, or the filler polynucleotide sequence can flank the 5′- or 3′-terminus of nucleic acid that encodes a protein or is transcribed into a transcript of interest.

Expression control elements include constitutive or regulatable control elements, such as a tissue-specific expression control element or promoter.

ITRs can be any of AAV2 or AAV6 or AAV8 or AAV9 serotypes, or a combination thereof. AAV particles can include any VP1, VP2 and/or VP3 capsid protein having 75% or more sequence identity to any of AAV1, AAV2, AAV3, AAV4, AAV5, AAV6, AAV10, AAV11, AAV-2i8, AAV rh74 or AAV 7m8 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, AAV-218, AAV rh74 and AAV 7m8 AAV serotypes.

Following production of recombinant viral (e.g., AAV) particles as set forth herein, if desired, the viral (e.g., rAAV) particles can be purified and/or isolated from host cells using a variety of conventional methods. Such methods include column chromatography, CsCl gradients, iodixanol gradient and the like.

For example, a plurality of column purification steps such as purification over an anion exchange column, an affinity column and/or a cation exchange column can be used. (See, e.g., WO 02/12455 and US 2003/0207439). Alternatively, or in addition, a iodixanol or CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAV vector production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV particle represents only a minor fraction of the biomass, rAAV particles need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

As an initial step, typically the cultivated cells that produce the rAAV particles are harvested, optionally in combination with harvesting cell culture supernatant (medium) in which the cells (suspension or adherent) producing rAAV particles have been cultured. The harvested cells and optionally cell culture supernatant may be used as is, as appropriate, lysed or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

Cells and/or supernatant of the harvest are lysed by disrupting the cells, for example, by chemical or physical means, such as detergent, microfluidization and/or homogenization, to release the rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (comprising rAAV vectors as well as empty capsids) and production/process related impurities, such as soluble cellular components from the host cells that can include, inter alia, cellular proteins, lipids, and/or nucleic acids, and cell culture medium components. The optionally clarified lysate is then subjected to purification steps to purify AAV particles (comprising rAAV vectors) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify rAAV particles.

A first chromatography step may be cation exchange chromatography or anion exchange chromatography. If the first chromatography step is cation exchange chromatography the second chromatography step can be anion exchange chromatography or size exclusion chromatography (SEC). Thus, in certain embodiments of all aspects and embodiments, rAAV particle purification is via cation exchange chromatography, followed by purification via anion exchange chromatography.

Alternatively, if the first chromatography step is cation exchange chromatography the second chromatography step can be size exclusion chromatography (SEC). Thus, in certain embodiments of all aspects and embodiments, rAAV particle purification is via cation exchange chromatography, followed by purification via size exclusion chromatography (SEC).

Still alternatively, a first chromatography step may be affinity chromatography. If the first chromatography step is affinity chromatography the second chromatography step can be anion exchange chromatography. Thus, in certain embodiments of all aspects and embodiments, rAAV particle purification is via affinity chromatography, followed by purification via anion exchange chromatography.

Optionally, a third chromatography can be added to the foregoing chromatography steps. Typically, the optional third chromatography step follows cation exchange, anion exchange, size exclusion or affinity chromatography.

Thus, in certain embodiments of all aspects and embodiments, rAAV particle purification is via cation exchange chromatography, followed by purification via anion exchange chromatography, followed by purification via size exclusion chromatography (SEC).

In addition, in certain embodiments of all aspects and embodiments, further rAAV particle purification is via cation exchange chromatography, followed by purification via size exclusion chromatography (SEC), followed by purification via anion exchange chromatography.

In yet further embodiments of all aspects and embodiments, rAAV particle purification is via affinity chromatography, followed by purification via anion exchange chromatography, followed by purification via size exclusion chromatography (SEC).

In yet further embodiments of all aspects and embodiments, rAAV particle purification is via affinity chromatography, followed by purification via size exclusion chromatography (SEC), followed by purification via anion exchange chromatography.

Cation exchange chromatography functions to separate the AAV particles from cellular and other components present in the clarified lysate and/or column eluate from an affinity or size exclusion chromatography. Examples of strong cation exchange resins capable of binding rAAV particles over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milliwaukee, WI, USA). Weak cation exchange resins include, without limitation, any carboxylic acid based resin. Exemplary cation exchange resins include carboxymethyl (CM), phospho (based on the phosphate functional group), methyl sulfonate(S) and sulfopropyl (SP) resins.

Anion exchange chromatography functions to separate AAV particles from proteins, cellular and other components present in the clarified lysate and/or column eluate from an affinity or cation exchange or size exclusion chromatography. Anion exchange chromatography can also be used to reduce and thereby control the amount of empty capsids in the eluate. For example, the anion exchange column having rAAV particle bound thereto can be washed with a solution comprising NaCl at a modest concentration (e.g., about 100-125 mM, such as 110-115 mM) and a portion of the empty capsids can be eluted in the flow through without substantial elution of the rAAV particles. Subsequently, rAAV particles bound to the anion exchange column can be eluted using a solution comprising NaCl at a higher concentration (e.g., about 130-300 mM NaCl), thereby producing a column eluate with reduced or depleted amounts of empty capsids and proportionally increased amounts of rAAV particles comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

The below outlined recombinant adeno-associated virus particle (rAAV particle) purification and production methods are scalable up to large scale. For example, to a suspension culture of 5, 10, 10-20, 20-50, 50-100, 100-200 or more liters volume. The recombinant adeno-associated virus particle purification and production methods are applicable to a wide variety of AAV serotypes/capsid variants.

In certain embodiments of all aspects and embodiments, the purification of rAAV particles comprises the steps of:
(a) harvesting cells and/or cell culture supernatant comprising rAAV particles to produce a harvest;
(b) optionally concentrating the harvest produced in step (a) to produce a concentrated harvest;
(c) lysing the harvest produced in step (a) or the concentrated harvest produced in step (b) to produce a lysate;
(d) treating the lysate produced in step (c) to reduce contaminating nucleic acid in the lysate thereby producing a nucleic acid reduced lysate;
(e) optionally filtering the nucleic acid reduced lysate produced in step (d) to produce a clarified lysate, and optionally diluting the clarified lysate to produce a diluted clarified lysate;
(f) subjecting the nucleic acid reduced lysate of step (d), the clarified lysate of step (e), or the diluted clarified lysate produced in step (e) to a cation exchange column chromatography to produce a column eluate comprising rAAV particles, thereby separating rAAV particles from protein impurities or other production/process related impurities, and optionally diluting the column eluate to produce a diluted column eluate;
(g) subjecting the column eluate or the diluted column eluate produced in step (f) to an anion exchange chromatography to produce a second column eluate comprising rAAV particles, thereby separating rAAV particles from protein impurities or production/process related impurities, and optionally concentrating the second column eluate to produce a concentrated second column eluate;
(h) subjecting the second column eluate or the concentrated second column eluate produced in step (g) to a size exclusion column chromatography (SEC) to produce a third column eluate comprising rAAV particles, thereby separating rAAV particles from protein impurities or production/process related impurities, and optionally concentrating the third column eluate to produce a concentrated third column eluate; and
(i) filtering the third column eluate or the concentrated third column eluate produced in step (h), thereby producing purified rAAV particles;
whereby the viral genome copy number is determined with the method according to the invention in or after one or more of steps (a) to (i).

In certain embodiments, steps (a) to (f) are maintained and combined with the following steps:
(g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a concentrated second column eluate;
(h) subjecting the second column eluate or the diluted second column eluate produced in step (g) to an anion exchange chromatography to produce a third column eluate comprising rAAV particles thereby separating rAAV particles from protein impurities production/process related impurities and optionally diluting the third column eluate to produce a diluted third column eluate; and
(i) filtering the third column eluate or the concentrated third column eluate produced in step (h), thereby producing purified rAAV particles;
whereby the viral genome copy number is determined with the method according to the invention in or after one or more of steps (a) to (i).

In certain embodiments, steps (a) to (g) are maintained and combined with the following step:
(h) filtering the second column eluate or the concentrated second column eluate produced in step (g), thereby producing purified rAAV particles;
whereby the viral genome copy number is determined with the method according to the invention in or after one or more of steps (a) to (h).

In embodiment, steps (a) to (e) are maintained and combined with the following steps:
(f) subjecting the nucleic acid reduced lysate in step (d), or clarified lysate or diluted clarified lysate produced in step (e) to an AAV affinity column chromatography to produce a column eluate comprising rAAV particles, thereby separating rAAV particles from protein impurities or other production/process related impurities, and optionally concentrating the column eluate to produce a concentrated column eluate;
(g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a diluted second column eluate;
(h) optionally subjecting the second column eluate or the diluted second column eluate produced in step (g) to an anion exchange chromatography to produce a third column eluate comprising rAAV particles, thereby separating rAAV particles from protein impurities or other production/process related impurities, and optionally diluting the third column eluate to produce a diluted third column eluate; and
(i) filtering the second column eluate or the diluted second column eluate produced in step (g), or filtering the third column eluate or the concentrated third column eluate produced in step (h), thereby producing purified rAAV particles;
whereby the viral genome copy number is determined with the method according to the invention in or after one or more of steps (a) to (i).

In certain embodiments of all aspects and embodiments, concentrating of step (b) and/or step (f) and/or step (g) and/or step (h) is via ultrafiltration/diafiltration, such as by tangential flow filtration (TFF).

In certain embodiments of all aspects and embodiments, concentrating of step (b) reduces the volume of the harvested cells and cell culture supernatant by about 2-20 fold.

In certain embodiments of all aspects and embodiments, concentrating of step (f) and/or step (g) and/or step (h) reduces the volume of the column eluate by about 5-20 fold.

In certain embodiments of all aspects and embodiments, lysing of the harvest produced in step (a) or the concentrated harvest produced in step (b) is by physical or chemical means. Non-limiting examples of physical means include microfluidization and homogenization. Non-limiting examples of chemical means include detergents. Detergents include non-ionic and ionic detergents. Non-limiting examples of non-ionic detergents include Triton X-100. Non-limiting examples of detergent concentration is between about 0.1 and 1.0% (v/v) or (w/v), inclusive.

In certain embodiments of all aspects and embodiments, step (d) comprises treating with a nuclease thereby reducing contaminating nucleic acid. Non-limiting examples of a nuclease include benzonase.

In certain embodiments of all aspects and embodiments, filtering of the clarified lysate or the diluted clarified lysate of step (e) is via a filter. Non-limiting examples of filters are those having a pore diameter of between about 0.1 and 10.0 microns, inclusive.

In certain embodiments of all aspects and embodiments, diluting of the clarified lysate of step (e) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, diluting of the column eluate of step (f) or the second column eluate of step (g) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, the rAAV particles resulting from step (i) are formulated with a surfactant to produce a rAAV particle formulation.

In certain embodiments of all aspects and embodiments, the anion exchange column chromatography of step (f), (g) and/or (h) comprises polyethylene glycol (PEG) modulated column chromatography.

In certain embodiments of all aspects and embodiments, the anion exchange column chromatography of step (g) and/or (h) is washed with a PEG solution prior to elution of the rAAV particles from the column.

In certain embodiments of all aspects and embodiments, the PEG has an average molecular weight in a range of about 1,000 g/mol to 80,000 g/mol, inclusive.

In certain embodiments of all aspects and embodiments, the PEG is at a concentration of about 4% to about 10% (w/v), inclusive.

In certain embodiments of all aspects and embodiments, the anion exchange column of step (g) and/or (h) is washed with an aqueous surfactant solution prior to elution of the rAAV particles from the column.

In certain embodiments of all aspects and embodiments, the cation exchange column of step (f) is washed with a surfactant solution prior to elution of the rAAV particles from the column.

In certain embodiments of all aspects and embodiments, the PEG solution and/or the surfactant solution comprises an aqueous Tris-HCl/NaCl buffer, an aqueous phosphate/NaCl buffer, or an aqueous acetate/NaCl buffer.

In certain embodiments of all aspects and embodiments, NaCl concentration in the buffer or solution is in a range of between about 20-300 mM NaCl, inclusive, or between about 50-250 mM NaCl, inclusive.

In certain embodiments of all aspects and embodiments, the surfactant comprises a cationic or anionic surfactant.

In certain embodiments of all aspects and embodiments, the surfactant comprises a twelve carbon chained surfactant.

In certain embodiments of all aspects and embodiments, the surfactant comprises Dodecyltrimethylammonium chloride (DTAC) or Sarkosyl.

In certain embodiments of all aspects and embodiments, the rAAV particles are eluted from the anion exchange column of step (f), (g) and/or (h) with an aqueous Tris-HCl/NaCl buffer.

In certain embodiments of all aspects and embodiments, the Tris-HCl/NaCl buffer comprises 100-400 mM NaCl, inclusive, optionally at a pH in a range of about pH 7.5 to about pH 9.0, inclusive.

In certain embodiments of all aspects and embodiments, the anion exchange column of step (f), (g) and/or (h) is washed with an aqueous Tris-HCl/NaCl buffer.

In certain embodiments of all aspects and embodiments, the NaCl concentration in the aqueous Tris-HCl/NaCl buffer is in a range of about 75-125 mM, inclusive.

In certain embodiments of all aspects and embodiments, the aqueous Tris-HCl/NaCl buffer has a pH from about pH 7.5 to about pH 9.0, inclusive.

In certain embodiments of all aspects and embodiments, the anion exchange column of step (f), (g) and/or (h) is washed one or more times to reduce the amount of empty capsids in the second or third column eluate.

In certain embodiments of all aspects and embodiments, the anion exchange column wash removes empty capsids from the column prior to rAAV particle elution and/or instead of rAAV particle elution, thereby reducing the amount of empty capsids in the second or third column eluate.

In certain embodiments of all aspects and embodiments, the anion exchange column wash removes at least about 50% of the total empty capsids from the column prior to rAAV particle elution and/or instead of rAAV particle elution, thereby reducing the amount of empty capsids in the second or third column eluate by about 50%.

In certain embodiments of all aspects and embodiments, the NaCl concentration in the aqueous Tris-HCl/NaCl buffer is in a range of about 110-120 mM, inclusive.

In certain embodiments of all aspects and embodiments, ratios and/or amounts of the rAAV particles and empty capsids eluted are controlled by a wash buffer.

In certain embodiments of all aspects and embodiments, the rAAV particles are eluted from the cation exchange column of step (f) in an aqueous phosphate/NaCl buffer, or an aqueous acetate/NaCl buffer. Non-limiting NaCl concentration in a buffer is in a range of about 125-500 mM NaCl, inclusive. Non-limiting examples of buffer pH are between about pH 5.5 to about pH 7.5, inclusive.

In certain embodiments of all aspects and embodiments, the anion exchange column of step (f), (g) and/or (h) comprises a quaternary ammonium functional group such as quaternized polyethylenimine.

In certain embodiments of all aspects and embodiments, the size exclusion column (SEC) of step (g) and/or (h) has a separation/fractionation range (molecular weight) from about 10,000 g/mol to about 600,000 g/mol, inclusive.

In certain embodiments of all aspects and embodiments, the cation exchange column of step (f) comprises a sulfonic acid or functional group such as sulphopropyl.

In certain embodiments of all aspects and embodiments, the AAV affinity column comprises a protein or ligand that binds to AAV capsid protein. Non-limiting examples of a protein include an antibody that binds to AAV capsid protein. More specific non-limiting examples include a single-chain Llama antibody (Camelid) that binds to AAV capsid protein.

In certain embodiments of all aspects and embodiments, the method excludes a step of cesium chloride gradient ultracentrifugation.

In certain embodiments of all aspects and embodiments, the method recovers approximately 50-90% of the total rAAV particles from the harvest produced in step (a) or the concentrated harvest produced in step (b).

In certain embodiments of all aspects and embodiments, the method produces rAAV particles having a greater purity than rAAV particles produced or purified by a single AAV affinity column purification.

In certain embodiments of all aspects and embodiments, steps (c) and (d) are performed substantially concurrently.

In certain embodiments of all aspects and embodiments, the NaCl concentration is adjusted to be in a range of about 100-400 mM NaCl, inclusive, or in a range of about 140-300 mM NaCl, inclusive, after step (c) but prior to step (f).

In certain embodiments of all aspects and embodiments, the cells are suspension growing or adherent growing cells.

In certain embodiments of all aspects and embodiments, the cells are mammalian cells. Non-limiting examples include HEK cells, such as HEK-293 cells, and CHO cells, such as CHO-K1 cells.

Methods to determine infectious titer of rAAV particles containing a transgene are known in the art (see, e.g., Zhen et al., Hum. Gene Ther. 15 (2004) 709). Methods for assaying for empty capsids and rAAV particles with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV particle can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

All references mentioned herein are incorporated herewith by reference.

The following examples are provided to aid the understanding of the present invention, the true scope of which is set forth in the appended claims. It is understood that modifications can be made in the procedures set forth without departing from the spirit of the invention.

### EXAMPLES

### General Techniques

### 1) Recombinant DNA Techniques

Standard methods are used to manipulate DNA as described in Sambrook et al., Molecular Cloning: A Laboratory Manual, Second Edition, Cold Spring Harbor Laboratory Press, Cold Spring Harbor, N.Y., (1989). The molecular biological reagents are used according to the manufacturer's instructions.

### 2) DNA and Protein Sequence Analysis and Sequence Data Management

The EMBOSS (European Molecular Biology Open Software Suite) software package, Invitrogen's Vector NTI and Geneious Prime and are used for sequence creation, mapping, analysis, annotation and illustration.

### 3) Gene and Oligonucleotide Synthesis

Desired gene segments are prepared by chemical synthesis at Geneart GmbH (Regensburg, Germany). The synthesized gene fragments are cloned into an E. coli plasmid for propagation/amplification. The DNA sequences of subcloned gene fragments are verified by DNA sequencing. Alternatively, short synthetic DNA fragments are assembled by annealing chemically synthesized oligonucleotides or via PCR. The respective oligonucleotides are prepared by metabion GmbH (Planegg-Martinsried, Germany).

### 4) Reagents

All commercial chemicals, antibodies and kits are used as provided according to the manufacturer's protocol if not stated otherwise.

### 5) Cloning

### General

For the plasmids, a cloning strategy via restriction enzymes was used. By selection of suitable restriction enzymes, the wanted gene of interest can be cut out and afterwards inserted into a different plasmid by ligation. Therefore, enzymes cutting in a multiple cloning site (MCS) are preferably used and chosen in a smart manner, so that a ligation of the fragments in the correct array can be conducted. If plasmid and fragment are previously cut with the same restriction enzyme, the sticky ends of fragment and plasmid fit perfectly together and can be ligated by a DNA ligase, subsequently. After ligation, competent E. coli cells are transformed with the newly generated plasmid.

### Cloning Via Restriction Digestion

For the digest of plasmids with restriction enzymes the following components are pipetted together on ice:

TABLE 11
Restriction Digestion Reaction Mix

| component | ng (set point) | μL |
|---|---|---|
| purified DNA | tbd | tbd |
| CutSmart Buffer (10x) | | 5 |
| Restriction Enzyme | | 1 |
| PCR-grade Water | | ad 50 |
| Total | | 50 |

If more enzymes are used in one digestion, 1 μL of each enzyme is used and the volume is adjusted by addition of more or less PCR-grade water. All enzymes are selected on the preconditions that they are qualified for the use with CutSmart buffer from new England Biolabs (100% activity) and have the same incubation temperature (all 37° C.).

Incubation is performed using thermomixers or thermal cyclers, allowing incubating the samples at a constant temperature (37° C.). During incubation the samples are not agitated. Incubation time is set at 60 min. Afterwards the samples are directly mixed with loading dye and loaded onto an agarose electrophoresis gel or stored at 4° C./on ice for further use.

A 1% agarose gel is prepared for gel electrophoresis. Therefore, 1.5 g of multi-purpose agarose are weighed into a 125 Erlenmeyer shake flask and filled up with 150 mL TAE-buffer. The mixture is heated up in a microwave oven until the agarose is completely dissolved. 0.5 μg/mL ethidium bromide are added into the agarose solution. Thereafter the gel is cast in a mold. After the agarose is set, the mold is placed into the electrophoresis chamber and the chamber is filled with TAE-buffer. Afterwards the samples are loaded. In the first pocket (from the left), an appropriate DNA molecular weight marker is loaded, followed by the samples. The gel is run for around 60 minutes at <130 V. After electrophoresis, the gel is removed from the chamber and analyzed in an UV-Imager.

The target bands are cut and transferred to 1.5 mL Eppendorf tubes. For purification of the gel, the QIAquick Gel Extraction Kit from Qiagen is used according to the manufacturer's instructions. The DNA fragments are stored at −20° C. for further use.

The fragments for the ligation are pipetted together in a molar ratio of 1:2, 1:3 or 1:5 plasmid to insert, depending on the length of the inserts and the plasmid-fragments and their correlation to each other. If the fragment, that should be inserted into the plasmid is short, a 1:5-ratio is used. If the insert is longer, a smaller amount of it is used in correlation to the plasmid. An amount of 50 ng of plasmid is used in each ligation and the particular amount of insert calculated with NEBioCalculator. For ligation, the T4 DNA ligation kit from NEB is used. An example for the ligation mixture is depicted in the following Table 12.

TABLE 12
Ligation Reaction Mix

| component | ng (set point) | conc. [ng/μL] | μL |
|---|---|---|---|
| T4 DNA Ligase Buffer (10x) | | | 2 |
| Plasmid DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components are pipetted together on ice, starting with the mixing of DNA and water, addition of buffer and finally addition of the enzyme. The reaction is gently mixed by pipetting up and down, briefly microfuged and then incubated at room temperature for 10 minutes. After incubation, the T4 ligase is heat inactivated at 65° C. for 10 minutes. The sample is chilled on ice. In a final step, 10-beta competent E. coli cells are transformed with 2 μL of the ligated plasmid (see below).

Transformation 10-Beta Competent E. coli Cells

For transformation, the 10-beta competent E. coli cells are thawed on ice. After that, 2 μL of plasmid DNA is pipetted directly into the cell suspension. The tube is flicked and put on ice for 30 minutes. Thereafter, the cells are placed into a 42° C. thermal block and heat-shocked for exactly 30 seconds. Directly afterwards, the cells are chilled on ice for 2 minutes. 950 μL of NEB 10-beta outgrowth medium are added to the cell suspension. The cells are incubated under shaking at 37° C. for one hour. Then, 50-100 μL are pipetted onto a pre-warmed (37° C.) LB-Amp agar plate and spread with a disposable spatula. The plate is incubated overnight at 37° C. Only bacteria, which have successfully incorporated the plasmid, carrying the resistance gene against ampicillin, can grow on these plates. Single colonies are picked the next day and cultured in LB-Amp medium for subsequent plasmid preparation.

### Bacterial Culture

Cultivation of E. coli is done in LB-medium, short for Luria Bertani, which is spiked with 1 mL/L 100 mg/mL ampicillin resulting in an ampicillin concentration of 0.1 mg/mL. For the different plasmid preparation quantities, the following amounts are inoculated with a single bacterial colony.

TABLE 13
E. coli cultivation volumes

| | Volume LB-Amp | Incubation |
|---|---|---|
| Quantity plasmid preparation | medium [mL] | time [h] |
| Mini-Prep 96-well (EpMotion) | 1.5 | 23 |
| Mini-Prep 15 mL-tube | 3.6 | 23 |
| Maxi-Prep | 200 | 16 |

For Mini-Prep, a 96-well 2 mL deep-well plate is filled with 1.5 mL LB-Amp medium per well. The colonies are picked and the toothpick is tuck in the medium. When all colonies are picked, the plate is closed with a sticky air porous membrane. The plate is incubated in a 37° C. incubator at a shaking rate of 200 rpm for 23 hours.

For Mini-Preps a 15 mL-tube (with a ventilated lid) is filled with 3.6 mL LB-Amp medium and equally inoculated with a bacterial colony. The toothpick is not removed but left in the tube during incubation. Like the 96-well plate, the tubes are incubated at 37° C., 200 rpm for 23 hours.

For Maxi-Prep 200 mL of LB-Amp medium are filled into an autoclaved glass 1 L Erlenmeyer flask and are inoculated with 1 mL of bacterial day-culture, that is roundabout 5 hours old. The Erlenmeyer flask is closed with a paper plug and incubated at 37° C., 200 rpm for 16 hours.

### Plasmid Preparation

For Mini-Prep, 50 μL of bacterial suspension are transferred into a 1 mL deep-well plate. After that, the bacterial cells are centrifuged down in the plate at 3000 rpm, 4° C. for 5 min. The supernatant is removed and the plate with the bacteria pellets is placed into an EpMotion. After approx. 90 minutes, the run is done and the eluted plasmid-DNA can be removed from the EpMotion for further use.

For Mini-Prep, the 15 mL tubes are taken out of the incubator and the 3.6 mL bacterial culture is splitted into two 2 mL Eppendorf tubes. The tubes are centrifuged at 6,800×g in a tabletop microcentrifuge for 3 minutes at room temperature. After that, Mini-Prep is performed with the Qiagen QIAprep Spin Miniprep Kit according to the manufacturer's instructions. The plasmid DNA concentration is measured with Nanodrop.

Maxi-Prep is performed using the Macherey-Nagel NucleoBond® Xtra Maxi EF Kit according to the manufacturer's instructions. The DNA concentration is measured with Nanodrop.

### Ethanol Precipitation

The volume of the DNA solution is mixed with the 2.5-fold volume ethanol 100%. The mixture is incubated at −20° C. for 10 min. Then the DNA is centrifuged for 30 min. at 14,000 rpm, 4° C. The supernatant is carefully removed and the pellet is washed with 70% ethanol. Again, the tube is centrifuged for 5 min. at 14,000 rpm, 4° C. The supernatant is carefully removed by pipetting and the pellet is dried. When the ethanol is evaporated, an appropriate amount of endotoxin-free water is added. The DNA is given time to re-dissolve in the water overnight at 4° C. A small aliquot is taken and the DNA concentration is measured with a Nanodrop device.

### Expression Cassette Composition

For the expression of an open reading frame, a transcription unit comprising at least the following functional elements is used:
a promoter,
a nucleic acid comprising the respective open reading frame including signal sequences, if required,
a polyadenylation signal sequence.

Beside the expression unit/cassette including the desired gene to be expressed, the basic/standard mammalian expression plasmid contains
an origin of replication from the plasmid pUC18 which allows replication of this plasmid in E. coli, and
a beta-lactamase gene which confers ampicillin resistance in E. coli.

### 6) Cell Culture Techniques

Standard cell culture techniques are used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Transient Transfections in HEK293 System

Cells producing a recombinant AAV particle have been generated by transient transfection with the respective plasmids using the HEK293 system (Invitrogen, now Thermo Scientific) according to the manufacturer's instruction. Briefly, HEK293 cells (Invitrogen) growing in suspension either in a shake flask or in a stirred fermenter in serum-free FreeStyle™ 293 expression medium (Invitrogen) are transfected with a mix of the respective plasmids and 293Fectin™ or fectin (Invitrogen). For 2 L shake flask (Corning) HEK293 cells are seeded at a density of 1*10⁶ cells/mL in 600 mL and are incubated at 120 rpm, 8% CO₂. The day after the cells are transfected at a cell density of ca. 1.5*10⁶ cells/mL with ca. 42 mL mix of A) 20 mL Opti-MEM (Invitrogen) with 600 μg total plasmid DNA (1 μg/mL) and B) 20 mL Opti-MEM+1.2 mL 293 fectin or fectin (2 μL/mL). According to the glucose consumption, glucose solution is added during the course of the fermentation.

### Example 1

### No Pre-Processing

### Procedure:

mix 90 μL H2O and 10 μL sample
incubate at 95° C. for 15 minutes

### Example

1. process sample
2. prepare PCR mastermix
3. add mastermix to plate
4. prepare 1:10 dilutions with water
5. add template to plate
6. seal plate and vortex (1 min at 2.200 rpm) and centrifuge
7. droplet formation (20 μL final mix+70 μL oil) and transfer to plate (42 μL) with automated droplet generator (Auto-DG)
8. seal plate and start PCR run

### Example 2

### Heat Denaturation

### Procedure:

incubate the sample at 98° C. for 10 minutes

### Example

1. heat denaturation
2. prepare PCR mastermix
3. add mastermix to plate
4. prepare 1:10 dilutions with water
5. add template to plate
6. seal plate and vortex (1 min at 2.200 rpm) and centrifuge
7. droplet formation (20 μL final mix+70 μL oil) and transfer to plate (42 μL) with Auto-DG
8. seal plate and start PCR run

### Example 3

### DNase I Digestion

### Reagents:

1) DNase I buffer (Promega): 400 mM Tris-HCl, pH 8, 100 mM MgSO₄, 10 mM CaCl₂)
2) DNase I (Promega): 50 U/mL diluted to 1 U/μL

### Procedure (50 μL Reaction Volume):

mix 30 μL H2O, 5 μL DNase I buffer, 5 μL DNase I, 10 μL sample
incubate at 37° C. for 30 min.
heat to 95° C. for 15 min.

### Procedure (100 μL Reaction Volume);

mix 75 μL H2O, 10 μL DNase I buffer, 5 L DNase I, 10 μL sample
incubate at 37° C. for 30 min.
heat to 95° C. for 15 min.

### EXAMPLE

1. DNase I digest;
2. add 50 μL water to the reaction mixture
3. prepare PCR mastermix
4. add PCR mastermix to plate (16.5 μL per well)
5. prepare 1:10 dilutions: 10 μL sample/plasmid/standard+90 μL H2O
6. add template to plate (5.5 μL per well)
7. seal plate and vortex (1 min at 2.200 rpm) and centrifuge (1 min. at 1000 rcf)
8. droplet formation (20 μL final mix+70 μL oil) and transfer to plate (42 μL) with Auto-DG
9. seal plate and start PCR run

### Example 4

### Proteinase K Digestion

### Reagents:

1) Proteinase K (Roche; 17.8 mg/mL=≥50 U/mL): diluted to 1 U/mL
2) proteinase K buffer (BioRad): 400 mM Tris-HCl, 20 mM EDTA, 2000 mM NaCl, pH 8
3) Sodium dodecyl sulfate solution (SDS-solution): 10%

### Procedure (Water):

mix 68 μL water with 10 μL sample and add 20 μL proteinase K
incubate at 50° C. for 60 minutes
heat to 95° C. for 15 minutes

### Procedure (Buffer Without SDS):

mix 63 μL water with 5 μL proteinase K buffer and add 10 μL sample as well as 20 μL proteinase K
incubate at 50° C. for 60 minutes
heat to 95° C. for 15 minutes
   Procedure (Buffer with SDS):
mix 53 μL water with 5 μL proteinase K buffer and add 10 μL SDS solution, 10 μL sample as well as 20 μL proteinase K
incubate at 50° C. for 60 minutes
heat to 95° C. for 15 minutes

### Example

1. Proteinase K digest
2. prepare PCR mastermix
3. add mastermix to plate (16.5 μL per well)
4. prepare 1:10 dilutions: 10 μL sample+90 μL H2O
5. add template to plate (5.5 μL per well)
6. seal plate and vortex (1 min. at 2.200 rpm) and centrifuge (1 min at 1000 rcf)
7. droplet formation (20 μL final mix+70 μL oil) and transfer to plate (42 μL) with Auto-DG
8. seal plate and start PCR run

### Example 5

### Heat Denaturation Followed by Proteinase K Digestion

### Reagents:

1) Proteinase K (Roche; 17.8 mg/mL=≥50 U/mL): 1 U/mL
2) proteinase K buffer (BioRad): 400 mM Tris-HCl, 20 mM EDTA, 2000 mM NaCl, pH 8

### Procedure:

heat denaturation: incubate the sample at 98° C. for 10 minutes
proteinase K digest: 1 μL proteinase K per 50 μL sample; incubate at 50° C. for 30 min.; inactivate at 95° C. for 10 min.

### EXAMPLE

1. heat denaturation
2. proteinase K digest
3. prepare 1:10 dilutions: 10 μL sample+90 μL H2O
4. prepare PCR mastermix
5. add mastermix to plate
6. add sample to plate
7. seal plate and vortex (1 min. at 2.200 rpm) and centrifuge
8. droplet formation (20 μL final mix+70 μL oil) and transfer to plate (42 μL) with Auto-DG
9. seal plate and start PCR run

### Example 6

### DNase I Digestion Followed by Proteinase K Digestion

### Method 1:

### Reagents:

1) DNase I buffer (Promega): 400 mM Tris-HCl, pH 8, 100 mM MgSO₄, 10 mM CaCl₂)
2) DNase I (Promega): 1 U/μL
3) Proteinase K (Roche; 17.8 mg/mL=≥50 U/mL): 1 U/mL
4) proteinase K buffer (BioRad): 400 mM Tris-HCl, 20 mM EDTA, 2000 mM NaCl, pH 8
5) Sodium dodecyl sulfate solution (SDS-solution): 10%

### Procedure:

mix 30 μL H2O, 5 μL DNase I buffer, 5 μL DNase I, 10 μL sample
incubate at 37° C. for 30 min.
heat to 95° C. for 15 min.
mix 50 μL PK-Mix (42 μL H2O+2 μL proteinase K+5 μL 20× proteinase K buffer+1 μL 10% SDS solution) with 50 μL incubated DNase I-Mix
incubate for 60 min. at 50° C.
heat to 95° C. for 15 min.

### Example

1. DNase I digest; optionally dilute 1:10
2. Proteinase K digest; dilute 1:10
3. prepare PCR mastermix
4. add PCR mastermix to plate (16.5 μL per well)
5. prepare 1:10 dilutions: 10 μL sample+90 μL H2O
6. add template to plate (5.5 μL per well)
7. seal plate and vortex (1 min, at 2.200 rpm) and centrifuge (1 min. at 1000 rcf)
8. droplet formation (20 μL final mix+70 μL oil) and transfer to plate (42 μL) with Auto-DG
9. seal plate and start PCR run

### Method 2:

### Reagents:

1) Proteinase K (NEB; approx. 20 mg/mL=≥800 U/mL): diluted to 16 U/mL
2) proteinase K buffer (BioRad): 400 mM Tris-HCl, 20 mM EDTA, 2000 mM NaCl
3) Sodium dodecyl sulfate solution (SDS-solution): 10%
   Procedure (Buffer with SDS):
mix 42 μL water with 50 μL sample (DNase I digestion solution) and add 2 μL proteinase K, 5 μL proteinase K buffer and 1 μL SDS solution
incubate at 50° C. for 60 minutes
heat to 95° C. for 15 minutes

### Example

1. DNase I digestion
2. proteinase K digestion
3. dilute 1:10
4. prepare PCR mastermix
5. add mastermix to plate (16.5 μL per well)
6. prepare 1:10 dilutions: 10 μL sample+90 μL H2O
7. add template to plate (5.5 μL per well)
8. seal plate and vortex (1 min. at 2.200 rpm) and centrifuge (1 min at 1000 rcf)
9. droplet formation (20 μL final mix+70 μL oil) and transfer to plate (42 μL) with Auto-DG
10. seal plate and start PCR run

### Example 7

### Comparison of Conditions

The reagents and procedures were as in the previous examples outlined.

The conditions were according to the following Tables 14 and 15.

TABLE 14

| condition | A | B | |
|---|---|---|---|
| enzyme | no DNase I | DNase I: 1 U/μL | |
| buffer | — | DNase I buffer (Promega) | |
| procedure | 90 μL H2O + 10 | 75 μL H2O + 10 μL buffer + | |
| | μL sample | 5 μL DNase I + 10 μL sample | |
| incubation temperature | — | 37° | C. |
| incubation time | — | 30 | min |

| | | |
|---|---|---|
| inactivation | 95° C. 15 min | 95° C. 15 min |

TABLE 15

| condition | W | X | | Y | | Z | |
|---|---|---|---|---|---|---|---|
| enzyme | no proteinase K (PK) | 1 U/mL proteinase K | | 1 U/mL proteinase K | | 1 U/mL proteinase K | |
| buffer | — | H2O | | proteinase K buffer | | proteinase K buffer | |
| | | | | | | 10% SDS | |
| procedure | 90 μL H2O + 10 μL | 68 μL H2O + 10 μL | | 63 μL H2O + 5 μL PK- | | 53 μL H2O + 5 μL PK- | |
| | sample | sample + 20 μL PK | | buffer + 10 μL sample + | | buffer + 10 μL 10% SDS + | |
| | | | | 20 μL PK | | 10 μL sample + 20 μL PK | |
| incubation | — | 50° | C. | 50° | C. | 50° | C. |
| temperature | | | | | | | |
| incubation time | — | 60 | min | 60 | min | 60 | min |

| | | | | |
|---|---|---|---|---|
| inactivation | 95° C. 15 min, 4° C. | 95° C. 15 min, 4° C. | 95° C. 15 min, 4° C. | 95° C. 15 min, 4° C. |

### Example 8

ddPCR

For viral genome titration, a duplexing ddPCR assay was performed. Primer and probes were designed against ITR sites and against the Amp resistance sequence, which is present on the backbone of all three plasmids used in the rAAV production. The PCR mastermix was prepared according to Table 16 (droplet digital PCR guide—Bio-Rad).

TABLE 16
ddPCR mastermix composition.

| | volume per | final |
|---|---|---|
| components | well [μL] | concentration |
| Supermix (2x) | 11 | 1x |
| 20 μM ITR primer fwd | 0.99 | 900 nM |
| 20 μM ITR primer rev | 0.99 | 900 nM |
| 20 μM FAM-labeled ITR probe | 0.275 | 250 nM |
| 20 μM Amp primer fwd | 0.99 | 900 nM |
| 20 μM Amp primer rev | 0.99 | 900 nM |
| 20 μM HEX-labeled Amp probe | 0.275 | 250 nM |
| template | 5.5 | 1*10E4−1*10E5 |
| | | copies/mL |
| water | 0.99 | — |
| total | 22 | |

The prepared mastermix was pipetted into a 96 well plate with 16.5 μL per well. Then, dilution series of the pretreated samples were conducted: 10 μL of samples were transferred with LoRentention Tips into 90 μL water in LoBind Tubes and thoroughly mixed. Thereafter, 5.5 μL of the samples were added to the mastermix solution in the 96 well plate in several dilution steps. The plate was sealed at 180° C., vortexed at 2,200 rpm for 1 min. and centrifuged at 1,000 rpm for another 1 min. With an automatic droplet generator device, which takes 20 μL PCR mix out of each well, up 20,000 droplets per well were produced and transferred into another 96 well plate. After sealing the droplet plate at 180° C., a PCR run was carried out. The respective conditions are shown in Table 17.

TABLE 17
ddPCR thermal cycling program.

| number of | | | final | |
|---|---|---|---|---|
| cycles | denaturation | annealing | elongation | end |
| 1 | 95° C., | | | |
| | 10 min | | | |
| 40 | 94° C., | 60° C., | | |
| | 30 sec | 1 min | | |
| 1 | | | 98° C., | 12° C., |
| | | | 10 min | maintained |

In a droplet reader, the fluorescence signal was measured for each droplet in the FAM and HEX channel. The QuantaSoft software processed the reader data and calculated copy numbers per 20 μL well for both target sequences, ITR sites and Amp. Initial sample titers can be determined with following equation 1:

$$copy number [ \frac{copies}{mL} ] = \frac{output [ \frac{copies}{20 μL well} ]}{5 [ \frac{μL sample}{20 μL well} ]} \cdot dilution factor \cdot 1000 [ \frac{μL}{mL} ] \qquad ( 2 )$$

### Example 9

rAAV Production

HEK293-F suspension cells were transfected with three plasmids, i.e. pAAV-transgene (EGFP or EBFP), pAAV-rep/cap and pAAV-helper. Plasmid DNA (1 μg/1 mL cell culture) and lipofection reagent PEI pro (2 μL/1 mL cell culture) were separately mixed with OptiMEM (50 μL/1 mL cell culture) (see, e.g., Grieger, J., et al. 2016). Afterwards, both solutions were combined, incubated at RT for 15 min. and added to HEK293-F cell suspension with 1*10E6 cell/mL in F17 medium. The cells were incubated at 37° C., 8% CO2, 120 rpm for 48 to 72 hours (Grieger, J., et al. (2016)).

Recombinant AAV particles were harvested by addition of a lysis buffer (100 μL/1 mL cell culture) containing 1% Triton X-100, 500 mM TRIS and 20 mM MgCl₂ at pH 7.5. Freshly diluted Benzonase was added (10 μL/1 mL cell culture) at a final concentration of 50 U/mL. After 60 min. lysis at 37° C. with agitation, MgSO₄ (final concentration 37.5 mM) was added and the cell lysis broth was incubated for another 30 min. (Chahal, P., et al. (2014)). Afterwards, the lysis suspension was centrifuged at 4,000 g for 20 min. and the supernatant was filtered through a 0.22 μm filter. The obtained product was considered as crude lysate.

### Example 10

rAAV Purification

A YMC glass column body was packed with POROS CaptureSelect AAVx affinity resin in a column bed volume of 9.1 mL. These resin beads are coated with an antibody fragment that binds a broad range of AAV serotypes with a high specificity (POROS CaptureSelect AAV Resins—User Guide 2017).

First, the column was equilibrated with phosphate buffered saline (PBS) to produce the right binding conditions. Then, the crude, filtered lysate was loaded with 150 cm/hour. After capturing the rAAV capsids, the column was washed with 4 column volumes (CV's) PBS, following 4 CV's 0.5 M NaCl to remove impurities like cell debris and DNA residues. Another wash step with 4 CV's PBS was performed to prepare for elution conditions (POROS CaptureSelect AAV Resins—User Guide 2017).

Afterwards, the rAAV capsids were eluted in 100 mM citric acid buffer (pH 2.4) (POROS CaptureSelect AAV Resins—User Guide 2017). Fractions within the elution peak (UV detection at λ=280 nm) were pooled. The pH-value was raised up to pH 7.5 using 2 M TRIS (pH 9). Finally, the eluate was sterile filtered using a syringe filter with a pore size of 0.2 μm.

1. A method for the determination of viral genome DNA copy number in a sample, wherein the method comprises the steps of
incubating the sample with proteinase K,
determining the viral genome DNA copy number by digital droplet polymerase chain reaction,
wherein the sample is free of DNA, which is not encapsidated within a viral particle,
wherein the incubation with proteinase K is in the presence of 0.05 (w/v) % to 1.5 (w/v) % sodium dodecyl sulfate.

2. The method according to claim 1, wherein the method comprises the following steps
incubating the sample with a nuclease to obtain a digested sample,
incubating the digested sample with proteinase K to obtain a proteinase K incubated sample,
determining the viral genome DNA copy number in the proteinase K incubated sample by digital droplet polymerase chain reaction.

3. The method according to claim 2, wherein the digested sample is diluted most 2.5-times for the incubation with proteinase K.

4. The method of claim 2, wherein the complete digested sample is incubated with proteinase K.

5. The method of claim 4, wherein the method is performed at a temperature of at most 95° C. except for the final elongation step of the digital droplet polymerase chain reaction.

6. The method of claim 4, wherein the total amount of proteinase K employed in the incubation is 15 mU to 35 mU.

7. The method of claim 6, wherein the volume of the sample is about 10 μL.

8. The method of claim 7, wherein the incubating with proteinase K is in a total volume of 100 μL.

9. The method of claim 2, wherein the nuclease is DNase I.

10. The method of claim 9, wherein the total amount of nuclease employed in the incubation is about 5 U.

11. The method of claim 10, wherein the incubating with the nuclease is in a total volume of 50 μL.

12. The method of claim 11, wherein the incubating with the nuclease is at final concentrations of 40 mM Tris*HCl, 10 mM MgSO₄, 1 mM CaCl₂) at a pH value of about 8.

13. The method of claim 12, wherein the incubating with the nuclease is at 37° C. for 30 minutes followed by an inactivating of the nuclease at 95° C. for 15 minutes.

14. The method of claim 13, wherein the incubating with proteinase K is at final concentrations of 20 mM Tris*HCl, 1 mM EDTA, 100 mM NaCl, 1 (w/v) % sodium dodecyl sulfate at a pH value of about 8.

15. The method of claim 13, wherein the incubating with proteinase K is at final concentrations of 30 mM Tris*HCl, 5 mM MgSO₄, 0.5 mM CaCl₂), 0.5 mM EDTA, 50 mM NaCl, 0.1 (w/v) % sodium dodecyl sulfate at a pH value of about 8.

16. The method of claim 15, wherein the incubating with proteinase K is at 50° C. for 60 minutes followed by an inactivating of the protease at 95° C. for 15 minutes.

17. The method of claim 16, wherein all steps of the method are performed at a temperature of at most 95° C. except for the final elongation step of the digital droplet polymerase chain reaction.
